(19) United States
(12) Patent Application Publication
(10) Pub. No.: US 2023/0160806 A1
(43) Pub. Date: May 25, 2023

(54) DEVICES AND METHODS FOR TWO-DIMENSION (2D)-BASED PROTEIN AND PARTICLE DETECTION

(71) Applicant: The Regents of the University of California, Oakland, CA (US)

(72) Inventors: Weian ZHAO, Irvine, CA (US); Enrico GRATTON, Irvine, CA (US); Per N. HEDDE, Irvine, CA (US)

(21) Appl. No.: 17/921,014

(22) PCT Filed: Apr. 23, 2021
(86) PCT No.: PCT/US2021/028778

§ 371 Date: Oct. 24, 2022

Related U.S. Application Data
(60) Provisional application No. 63/014,845, filed on Apr. 24, 2020.

(51) Int. Cl. G01N 15/14; G02B 21/08; G02B 21/00; G02B 21/16; G02B 21/36; G01N 33/543; G06K 7/14; G06K 7/10

(52) U.S. Cl. CPC G01N 15/1434; G01N 15/1404; G02B 21/086; G02B 21/0008; G02B 21/16; G02B 21/361; G01N 33/54306; G06K 7/1413; G06K 7/10722; H04N 25/71

(57) ABSTRACT

Provided are processes, methods, kits, devices and software for testing and detecting proteins such as antigens, cytokines or antibodies, particles or cells in specimens of or samples from human or animals; and in alternative embodiments the protein are induced by or derived from viruses, bacteria, an immune system, a cancer cell or any cell which can cause a disease, infection or condition such as a COVID-19 infection. Provided are portable imaging systems comprising flat static surfaces or slides, wherein the flat static surfaces or slides can be fabricated as printed microarrays, or biochips that can support protein or bioparticle precipitates. Provided are portable imaging systems comprising imaging systems with light sheet illumination to image two dimensional (2D) planes in liquids to detect proteins, bioparticles, cells, and organisms. Portable imaging systems provided herein can be used for point-of-care diagnosis, immunity analysis, epidemiological surveillance, and therapeutics and vaccine development.

### RELATED APPLICATIONS

This Patent Convention Treaty (PCT) International Application claims the benefit of priority to U.S. Provisional Application Serial No. (USSN) 63/014,845, Apr. 24, 2020. The aforementioned application is expressly incorporated herein by reference in its entirety and for all purposes.

### STATEMENT AS TO FEDERALLY SPONSORED RESEARCH

This invention was made with government support under National Institutes of Health (NIH), DHHS, grant nos. 1 R01 AI117061 and P41-GM103540. The government has certain rights in the invention.

### TECHNICAL FIELD

This invention generally relates to processes, methods and devices for protein and particle detection, testing or to analyze markers in patient specimens and samples such as saliva, sputum, urine, blood or blood components (for example, serum, plasma) for investigating biology and for clinical diagnosis and public health applications. In alternative embodiments, provided are processes, methods, kits, devices and software for testing and detecting proteins such as antigens, cytokines or antibodies, particles or cells in specimens of or samples from human or animals; and in alternative embodiments the protein are induced by or derived from viruses, bacteria, an immune system, a cancer cell or any cell which can cause a disease, infection or condition such as a COVID-19 infection. In alternative embodiments, provided are portable imaging systems comprising flat static surfaces or slides, wherein the flat static surfaces or slides can comprise or be fabricated as printed microarrays, biochips, protein precipitates, beads or high throughput imaging systems of 2D planes in liquids. In alternative embodiments, portable imaging systems as provided herein can enable point-of-care diagnosis, immunity analysis, epidemiological surveillance, and/or therapeutics and vaccine development.

### BACKGROUND

In recent years, mobile electronic devices such as smart phones, single board computers, and wearables have become more sophisticated and advanced. These devices feature a variety of sensors such as high-resolution cameras, are equipped with the latest communication technology including wireless data transfer, and possess computational power exceeding previously available desktop computers. Due to these properties, portable devices have great potential in biomedical applications allowing fast, inexpensive on-site biodetection and bioanalysis, especially using imaging-based methods.

Various adapters for microscopy using portable devices such as cell phones have been developed. In mobile devices, different illuminations strategies have been reported including on-axis epi-illumination [1], off-axis inclined illumination [2], butt-coupling [3], and total internal reflection [4]. In order to avoid out-of-focus background with these illumination schemes, either the sample is compressed to a thickness of approximately 10 μm by mounting it between two glass slides [5] or physical properties of the sample such as plasmonic enhancement due to the presence of a metal surface [2] or total internal reflection due to the presence of a refractive index change are exploited [4].

Protein and biological particles (for example, cells, bacteria, viruses) detection is of great importance in research and clinical diagnostics. Numerous assay platforms are available including, for example, immunohistochemistry (IHC), enzyme linked-immunosorbent assay (ELISA), flow cytometry, mass spectrometry, lateral flow test, chemiluminescent immunoassay, and other types of immunoassays. Protein and particle detection assay formats often involve capturing the target analytes on a solid surface such as an array or bead, followed by subsequent staining steps with, for example, luminophores to “light up” the target, prior to analysis. For particles such as a cell or proteins associated with a cell, they can be directly marked, without a solid surface support, using luminescent probes. An exemplary format of protein detection assay comprises a microarray that are coated with cognate biological molecules to detect target proteins such as antibodies in serology or a serological assay or detect multiple isotypes against tens, hundreds or thousands of antigens in a high throughput manner. Because multiple targets are incorporated in the microarray, it can achieve very high specificity and readily distinguish one disease marker from another. One such example that is of clinical interest is to discriminate different respiratory infections, for example diagnosing SARS-CoV-2 infection from Severe acute respiratory syndrome (SARS), Middle East respiratory syndrome (MERS), Flu, and other common coronaviruses. One such example of antigen microarrays for SARS-CoV-2 detection is reported by Khan and Assis [6,7]. They demonstrated a coronavirus antigen microarray that included antigens from SARS-CoV-2 and tested it on human sera collected prior to the pandemic to demonstrate low cross-reactivity with antibodies from human coronaviruses that cause the common cold, particularly for the S1 domain. Microarrays can also be used for low cost, high throughput testing on the scale of greater than 100,000 samples, which is critical to (repeatedly) test large populations. While biomarker labeling and assay chemistries are generally established, a major remaining roadblock in protein and biological particle detection is the lack of portable, easy-to-use, cost-effective yet high quality imaging and analysis modules. For instance, while microarrays and microbeads can be assayed with a minimal number of reagents and a simple infrastructure within minutes to hours, reading the stained specimen (for example, microarray slides after staining) by fluorescence imaging currently requires expensive ($10,000 or more) and sophisticated instruments that are not currently equipped in many clinics, hospitals and testing labs and are difficult to move to mobile testing sites such as drive through and field clinics. For instance, current microarray imaging systems comprise large laser scanners that depend on high precision 2D movement of the slide (or optical apparatus), a high-power laser and PMT detectors. Due to their outdated complexity, laser scanners often cost $50,000-$100,000. There are camera-based imagers available, however, despite their simpler designs, these devices are still pricy at $10,000-$30,000, power inefficient, too complex, large and heavy. These devices are also restricted to using a specific format such as a slide of fixed dimensions and are not compatible with randomly oriented samples and detection within liquids. In order to widely test a large population for a disease, and generally for bioanalysis purposes, portable, easy-of-use, and inexpensive imagers or analyzers need to be developed and integrated with protein and particle detection methods including for example microarray and microbead assays.

### SUMMARY

In alternative embodiments, provided are products of manufacture fabricated or manufactured as portable devices equipped with sensors, which can be inexpensive but powerful, such as for example a camera, paired with on-device and an online data processing. These exemplary products of manufacture have great potential in providing point-of-care high accuracy diagnosis and greatly improve human health related aspects, especially in the presence of epidemics/pandemics. Products of manufacture as provided herein can enable large scale, high throughput imaging of two-dimensional (2D) planes on substrates such as microarrays or microwells, and also within liquids such as contained in a cuvette, a capillary or a flow cell, relevant to antibody testing with (fluorescence) detection based on use of cameras, for example, mobile device cameras. In alternative embodiments, the easy-to-manufacture, low-cost portable devices as provided herein enable and use epi-illumination or light sheet illumination and imaging of surfaces and liquids in one or multiple spectral windows. Geared towards large scale protein detection such as antibody testing including viruses such as SARS-CoV-2, products of manufacture as provided herein can be very powerful for protecting the public's health.

In alternative embodiments, a product of manufacture, a device, an apparatus or a system for testing for the presence of a target analyte in a sample, as provided herein: is cheaper than most comparable devices; is portable; combines large, uniform field of view (optionally a field of view greater than (>) about 10 mm to about 20 mm) with high spatial resolution (and optionally the high spatial resolution is better than about 10 μm); provides fast imaging speed (optionally the acquisition time is less than (<) about 1 second (s) per image; and/or provides multichannel acquisition (and optionally the multichannel acquisition is greater than (>) about 2 or 3 channels.

In alternative embodiments, provided are products of manufacture, devices, apparatus or systems for testing for the presence of a target analyte in a sample,
wherein optionally the system is a multiplexed system,
and optionally the sample is derived or taken from an individual in need thereof,
and optionally the target analyte comprises a disease biomarker, a protein, a cytokine, a biological particle or a cell,
and optionally the disease biomarker, protein, cytokine or biological particle is expressed by or on or is derived from a cell,
and optionally the target analyte comprises at least one or a plurality of viral or microbial antigens, optionally coronavirus antigens, optionally SARS-CoV-2 antigens or antigen variants,
and optionally the sample comprises a biological or clinical sample, and optionally the biological or clinical sample comprises a liquid sample, and optionally the liquid sample comprises a serum sample, the product of manufacture, device, apparatus or system comprising:
(a) a functionalized surface, an assay surface or a sample slide,
   wherein optionally the functionalized surface or sample slide comprises or is fabricated as a biochip, a microarray or an equivalent, or the functionalized surface is on or fabricated as a bead or a microbead or an equivalent,
   and optionally the functionalized surface or sample slide comprises an assay surface that displays or has affixed thereon or carries: at least about 2, at least about 3, at least about 4, at least about 5, at least about 6, at least about 10, at least about 20, at least about 50, at least about 100, at least about 200, at least about 1,000, at least about 10,000, or more, discrete areas or spots, and each discrete area or spot comprises, displays, has affixed thereon or carries: a distinct target analyte-binding composition, or a plurality of compositions each capable of specifically binding to a specific target analyte,
   wherein optionally the distinct target analyte-binding composition comprises a protein, and optionally the protein comprises an antibody or antigen binding fragment thereof;
(b) an imaging system capable of imaging a sample in a solution or on a hard surface comprising:
(i) a sample illumination or sample luminescence, fluorescence or phosphorescent excitation device capable of or fabricated to produce an excitation light or excitation beam,
   wherein optionally the sample illumination or sample luminescence, fluorescence or phosphorescent excitation device comprises or is fabricated as an epi-illumination-based slide imager, or, a light sheet-based solution imager;
   wherein optionally an illumination pattern made by the sample illumination or the sample luminescence, fluorescence or phosphorescent excitation device comprises a round or a rectangular shape to provide uniform epi-illumination of a surface area,
   and optionally the surface area is about 5×5 mm to about 50×50 mm, or, in a solution, the illumination pattern is in the form of a light sheet between about 1 μm to about 15 μm thickness to provide optical sectioning, and optionally to provide optical sectioning in an area of about 0.5×0.5 mm to about 5×5 mm,
(ii) a camera or equivalent or a two-dimensional (2D) sensor or equivalent capable of focusing on or capturing images of or detecting light emitted from the two-dimensional (2D) sensor or equivalent and the functionalized surface, an assay surface or a sample slide;
   and optionally the 2D sensor or equivalent is a high resolution sensor, and optionally the high resolution sensor has a capability of about 1,000×1,000 pixels to about 10,000×10,000 pixels,
   and optionally the 2D sensor or equivalent is a monochrome or color complementary metal-oxide-semiconductor (CMOS) sensor, and optionally the CMOS sensor provides frame rates of about 1 frame per second to about 100 frames per second,
(iii) one or a plurality of lenses for image magnification and focusing of the excitation beam aligned between the camera or equivalent or the two-dimensional (2D) sensor or equivalent and the functionalized surface, an assay surface or a sample slide;
one or a plurality of optical filters to define excitation and detection spectral windows; and,
(iv) a sample holder,
wherein the functionalized surface or sample slide is loaded or positioned onto or into the sample holder or cuvette or equivalent,
and the sample illumination or sample luminescence, fluorescence or phosphorescent excitation are provided by one or multiple light sources in the form of epi-illumination or in the form of a light sheet,
and luminescent, fluorescence or phosphorescent images are detected with the camera or 2D sensor equivalent,
   wherein optionally the sample holder comprises or is fabricated as a slide holder or a cuvette or equivalent,
   and optionally the excitation light has between about 1 to 100 nm bandwidth,
   and optionally the excitation light bandwidth is a narrowband laser light, wherein optionally the narrowband laser light has about a less than (<) 5 nm bandwidth,
   and optionally the excitation light comprises a light-emitting diode (LED) light or a light emitting diode,
   and optionally the LED light(s) or light emitting diode(s) emit in a specific wavelength range, and optionally the specific wavelength range is in a wavelength range of between about 460 nm to 470 nm, 520 nm to 530 nm or 620 nm to 630 nm,
   and optionally emitters of different wavelengths are combined in the same light emitting diode, and optionally the light emitting diode comprises or is a Red, Green, Blue (RGB) emitter,
   and optionally the LED light or light emitting diode comprises one or a plurality of spectral filters,
   and optionally the one or the plurality of spectral filters have a bandwidth of between about 5 nm to about 100 nm (to result in a narrower spectral band),
   and optionally the camera or 2D sensor is operatively connected to a lens or system (or plurality) of lenses to relay and magnify the image,
   and optionally the camera or 2D sensor is operatively coupled to one or multiple (or plurality of) optical filters to define or to narrow detection of (or detected) spectral band or bands,
   and optionally the camera or 2D sensor or equivalent is or is fabricated as a mobile camera or a 2D sensor equivalent device, or a dedicated camera or 2D sensor equivalent,
and the camera or 2D sensor equivalent is operatively connected to a computer, a smartphone, or equivalent,
and the imaging system, or the camera or 2D equivalent, is operatively linked to a firmware and/or software or computer program to store, process and/or transmit data representing images from the camera or 2D equivalent; and
(c) a firmware and/or software or computer program to acquire, store and process images from the imaging system in a manner suitable for storing, identifying, quantifying and optionally classifying target analytes.

In alternative embodiments, a product of manufacture, device, apparatus or system as provided herein further comprises: (d) a software and/or a computer program to visualize, analyze, share and/or store data representing the images, and optionally the software and/or the program visualizes, analyzes, shares and/or stores the data on the product of manufacture, device, apparatus or system itself or by connecting the product of manufacture, device, apparatus or system to a server or cloud computing-based storage and/or sharing system.

In alternative embodiments, a product of manufacture, device, apparatus or system as provided herein comprises an about 0.2 to 200 megapixels charge-coupled device (CCD) or a Complementary Metal Oxide Semiconductor (CMOS) color or monochrome camera coupled to a lens or lens system of between about 1 mm to about 1400 mm focal length to yield a field of view of between about 1 mm to about 75 mm with a pixel size at the sample of between about 0.1 μm to 100 μm for 2D imaging of: surfaces with epi-illumination, or solutions with light sheet illumination, and optionally a fluorescent, luminescent or phosphorescent light is filtered with a long pass, band pass or short pass emission filter to prevent excitation light from reaching the camera sensor.
and optionally the emission light is separated with multiple optical filters into several color channel for multiplex imaging, and optionally one or multiple of the optical filters have a transmission response in the shape of a sine or cosine function or linear ramp to spectrally resolve the detected signal,
and optionally the emission light is separated with a dispersive element into a plurality of spectral channels to spectrally resolve the signal,
and optionally the dispersive element comprises a prism or a grating,
and optionally the emission light is separated with a dispersive element into between about 4 and about 1,000 spectral channels,
and optionally the excitation light is modulated and the emission light is detected in a time-resolved fashion (demodulated) to separate components of different luminescence, fluorescent or phosphorescent lifetimes;
wherein the slide holder or cuvette or equivalent comprises a slot or slide insert to accurately position the sample slide, biochip or microarray for imaging,
wherein optionally the slide holder or cuvette or equivalent can accurately position the slide, biochip or microarray at a precision higher than about 1 mm,
and optionally the sample slide, biochip, microarray or cuvette or equivalent can be contained in a cassette or sled to adapt to contain or hold slides, biochips, microarrays, cuvette or equivalent of different sizes to the same imaging device,
and optionally the sample slide, biochip, microarray or cuvette or equivalent have dimensions of between about 1 mm to 5 mm to about 100 mm to 120 mm,
and optionally a cassette or sled is used to position and image between about 1 to about 20 slides at a time on a single device,
and optionally a cassette or sled is used to position and image between about 1 to about 20 slides in between about 1 to 15 min,
and optionally the sample slide, biochip, microarray or cuvette or equivalent is moved through the imaging device either manually, semi-automated and/or fully automated.

In alternative embodiments of a product of manufacture, device, apparatus or system as provided herein, the sample slide is illuminated by one or more light sources from the top, bottom and/or side at an angle of between about 0° to 90° to the optical axis or with a light sheet at an angle of between about 60° to 120° to the signal detection axis with the one or multiple light sources,
and optionally the one or multiple light sources comprise or emit visible, near ultraviolet (UV) and/or near infrared (IR) light,
and optionally the visible light comprises light at between about 400 nm to about 700 nm, the near UV comprises light at between about 250 to about 400 nm, and/or near IR comprises light at between about 700 to about 1300 nm,
and optionally the one or multiple light sources are generated using one or more LEDs and/or laser diodes.

In alternative embodiments of a product of manufacture, device, apparatus or system as provided herein, illumination comprises or the light source emits epi-illumination, or the illumination comprises or the light source emits light in the form of a sheet or single plane of light at the sample plane,
wherein optionally the sheet or single plane of light at the sample plane is between about 1 μm to 15 μm in thickness,
and optionally the illumination is formed with a cylindrical optical element or by beam scanning,
and optionally the illumination comprises broad band excitation light that is spectrally cleaned with one or more optical filters,
and optionally the one or more optical filters comprises short pass or band pass filters,
and optionally the short pass or band pass filters have between about a 1 nm to about 100 nm bandwidth,
and optionally the source of the illumination or the light source comprises one or multiple LEDs or lasers,
and optionally between about 1 to 100 LEDs or lasers are used,
and optionally between about 1 to 100 LEDs or lasers with different wavelengths are used for multiplex acquisitions in multiple color channels,
and optionally between about 1 to 20 or 2 to 10 different wavelengths are emitted by the LEDs or lasers,
and optionally the light source and/or detector are modulated or pulsed for time-resolved signal detection to increase sensitivity or multiplexing applications,
and optionally time-resolved measurements comprise fluorescence and/or phosphorescence lifetime detection,
and optionally a modulation/pulse frequency for fluorescence lifetime detection is between about 1 to about 100 MHz,
and optionally a modulation/pulse frequency for phosphorescence lifetime detection is between about 1 Hz to about 1 MHz.

In alternative embodiments of a product of manufacture, device, apparatus or system as provided herein, the components of the imaging system are designed or fabricated to image surfaces, slides, biochips, microarrays or other target analytes in a sample or solution stained with a detectable dye or particle,
and optionally the detectable dye or particle comprises a fluorescent dye or particle,
and optionally the detectable dye or particle is conjugated to an antibody,
and optionally the antibody comprises a detecting antibody, a secondary antibody or an antibody comprising a streptavidin/biotin linkage,
and optionally the detectable dye or particle comprises: DyLight 405, Cy2, Cy3, Cy5, Alexa488, Alexa546, Alexa594, Alexa647, Atto488, Atto550, Atto647 or an equivalent or a combination thereof.
and optionally the detectable particle comprises: a QD525, QD565, QD585, QD605, QD655, QD705, QD800 nm emission ultrabright fluorescent microsphere or an equivalent or a combination thereof.

In alternative embodiments of a product of manufacture, device, apparatus or system as provided herein, the firmware and/or software or computer program used to acquire, store and process images from the imaging system in a manner suitable for storing, identifying, quantifying and optionally classifying target analytes comprises a firmware and/or computer software to interface with the camera or 2D imaging equivalent,
and optionally the firmware and/or software comprises an element or is able to: adjust the camera exposure time and/or sensitivity setting, and/or to correct for image distortions by referencing with an internally stored calibration pattern, to subtract background to eliminate spatial variations of the signal offset, and/or to find the position of each spot of a microarray image by aligning to reference markers printed on the microarray slides, and/or to optimize the dynamic range and ensure a linear response,
and optionally the camera is adjusted to have an exposure time between about 1 microsecond (μs) and 10 seconds (s),
and optionally the camera is adjusted to have a sensitivity setting between about ISO 64 to about ISO 128,000.

In alternative embodiments of a product of manufacture, device, apparatus or system as provided herein, a barcode attached or printed on the sample slide or cuvette or equivalent,
and optionally the barcode is read with the camera or with an external reader,
and optionally the barcode read is stored with an image or images of each sample for unambiguous sample identification.

In alternative embodiments of a product of manufacture, device, apparatus or system as provided herein:
a wired (optionally Ethernet, USB) or wireless connection (optionally Bluetooth, WiFi or equivalents) or equivalent is used to transfer image data to an online server and/or a cloud-based system;
the processing software comprises data comprising the computation of fluorescence, luminescence or phosphorescence intensity values for each spot or particle on a surface or in a 2D plane within a solution by calculating the mean or median value of the intensity distribution within the spot area,
   and optionally the calculating comprises referencing the data to a known antigen layout of the microarray, biochip or sample slide or composition of the capture beads or microbeads,
   and optionally attached dyes with known concentrations as internal reference, probability values (p values) for the presence of target analytes specific to a disease can be calculated,
   and optionally the referencing step is carried out either on the device itself or on a server/cloud-based system after data upload;
the distinct target analyte comprises a distinct protein,
   and optionally distinct protein is an antibody or an antigen,
   and optionally the antigen is derived from a microorganism, and optionally the microorganism is a bacterium or a virus, and optionally the viral antigen comprises a SARS-CoV-2 antigen or antigenic variant,
   and optionally the antibody specifically binds to a microorganism, and optionally the microorganism is a bacterium or a virus, and optionally the viral antigen comprises a SARS-CoV-2 antigen or antigenic variant,
   and optionally the functionalized surface comprises, displays or carries at least 1 coronavirus antigen, and optionally the coronavirus comprises an S protein antigen,
   and optionally the coronavirus is or comprises SARS-CoV or MERS-CoV,
   and optionally the functionalized surface comprises, displays or carries a non-coronavirus antigen, optionally a viral or a bacterial antigen,
   and optionally the viral antigen is or comprises a HKU1, OC43, NL63, 229E or influenza antigen;
the target analytes are captured or affixed on a surface or the sample slide,
   wherein optionally the microarray, biochip, sample slide or bead or microbead is probed with a detectable dye or a nanoparticle, optionally using a sandwich assay,
   and optionally the bead or microbead with a captured target analyte is subsequently immobilized on a surface, or a slide, or a microwells, or a microchamber, prior to imaging,
   and optionally the bead or microbead with the captured target analytes remains suspended in solution for imaging;
a target analyte is directly labeled with a detectable dye or a nanoparticle prior to immobilizing or affixing onto a surface for analysis, or the target analyte is analyzed directly in solution,
wherein optionally the target analyte is or is derived from: a cell, a cancer cell, a CTC, an immune cell, a bacterium, a yeast cell, a pathogen, a virus or a parasite; and/or,
the product of manufacture, device, apparatus or system is fabricated as a portable device.

In alternative embodiments of a product of manufacture, device, apparatus or system as provided herein, the device further comprises or is fabricated to have:

a hollow tubing for flowing or moving a liquid sample, wherein the hollow tubing is operatively linked to a transparent sample flow cell, a pump and optionally an automated axial sample holder, wherein a liquid sample flows through the hollow tubing, the transparent sample flow cell and the pump;

a pump operatively linked to the hollow tubing, wherein flow of a liquid in and through the hollow tubing and the transparent sample flow cell is controlled and driven by the pump,

an automated axial sample holder and a mechano-electric component, wherein movement of the automated axial sample holder is controlled by the mechano-electric component, and the automated axial sample holder positions the transparent sample flow cell before a source of light or illumination or an excitation light or excitation beam,

wherein optionally the automated axial sample holder comprises or is fabricated as a piezo actuator or voice coil actuator;

and optionally the transparent sample flow cell is or is fabricated as a cuvette or equivalent,

and optionally the source of light or illumination is or is fabricated as a sample illumination or sample luminescence, fluorescence or phosphorescent excitation device capable of or fabricated to produce an excitation light or excitation beam,

and optionally in combination with flowing the liquid solution through the transparent sample flow cell (optionally a cuvette or equivalent) a volume of liquid (optionally between about 0.1 ml to about 10 ml liquid) is imaged (optionally in a few minutes) with the ability to detect the presence of a particle (optionally able to detect only a single particle).

In alternative embodiments, provided is a product of manufacture, device, apparatus or system for use in detecting one or a plurality of biomarkers, optionally one or a plurality of proteins, optionally one or a plurality of viral or a microbial antigens, optionally one or a plurality of antibodies, optionally one or a plurality of cytokines, optionally one or a plurality of biological particles, optionally one or a plurality of cells, or diagnosing, or optionally measuring immune response to, a viral or a microbial infection, cancer, autoimmune disorder, or inflammation comprising use of a product of manufacture, device, apparatus or system as provided herein.

In alternative embodiments, provided are uses of a product of manufacture, device, apparatus or system for detecting one or a plurality of biomarkers, optionally one or a plurality of proteins, optionally one or a plurality of viral or a microbial antigens, optionally one or a plurality of antibodies, optionally one or a plurality of cytokines, optionally one or a plurality of biological particles, optionally one or a plurality of cells, or diagnosing, or optionally measuring immune response to, a viral or a microbial infection, cancer, autoimmune disorder, or inflammation, wherein the product of manufacture, device, apparatus or system is a product of manufacture, device, apparatus or system as provided herein.

In alternative embodiments, provided are methods for detecting one or a plurality of biomarkers, optionally one or a plurality of proteins, optionally one or a plurality of viral or a microbial antigens, optionally one or a plurality of antibodies, optionally one or a plurality of cytokines, optionally one or a plurality of biological particles, optionally one or a plurality of cells, or diagnosing, or optionally measuring immune response to, a viral or a microbial infection, cancer, autoimmune disorder, or inflammation, comprising:

(a) providing of having provided a product of manufacture, device, apparatus or system as provided herein;

(b) contacting the product of manufacture, device, apparatus or system with a biological sample or a sample derived from a biological source comprising a target analyte,

wherein optionally the biological sample or the sample derived from the biological source comprising a target analyte comprises or is derived from a solid tissue sample, a biopsy or a biological liquid, wherein optionally the biological liquid comprises or is derived from a serum, blood, urine, cerebral spinal fluid, mucous or sputum sample;

(c) determining if the biological sample or the sample derived from the biological source comprises a composition (optionally a protein) that specifically binds to a target analyte affixed on the product of manufacture, device, apparatus or system.

In alternative embodiments of methods as provided herein, the target analyte comprises or is a substantially stained or detectably labeled target analyte,
wherein optionally the stained or detectably labeled target analyte comprises a fluorescently or luminescently stained target analyte, and optionally the stained or detectably labeled target analyte is in solution or in a liquid form,
and optionally target analytes in a sample or solution are stained with a detectable dye or particle before the target analyte is contacted with or to the product of manufacture, device, apparatus or system,
and optionally the detectable dye or particle comprises a fluorescent dye or particle,
and optionally the detectable dye or particle is conjugated to an antibody,
and optionally the antibody comprises a detecting antibody, a secondary antibody or an antibody comprising a streptavidin/biotin linkage,
and optionally the detectable dye or particle comprises: DyLight 405, Cy2, Cy3, Cy5, Alexa488, Alexa546, Alexa594, Alexa647, Atto488, Atto550, Atto647 or an equivalent or a combination thereof.
and optionally the detectable particle comprises: a QD525, QD565, QD585, QD605, QD655, QD705, QD800 nm emission ultrabright fluorescent microsphere or an equivalent or a combination thereof.

In alternative embodiments of methods as provided herein: the determining if the biological sample or the sample derived from the biological source comprises using a composition (optionally a protein) that specifically binds to a specific target analyte affixed on the product of manufacture, device, apparatus or system, and the composition comprises a substantially stained or a detectable agent or moiety that can specifically bind to a target analyte that is specifically bound to an assay surface of the product of manufacture, device, apparatus or system, wherein optionally the composition is detectably (optionally fluorescently) stained, and optionally the composition is in solution or in a liquid form.

In alternative embodiments, provided are methods for treating or ameliorating a disease, a condition, or a viral or a microbial infection, comprising:

(a) testing or screening an individual in need thereof with a product of manufacture, device, apparatus or system as provided herein, to determine if the individual in need thereof is infected with a pathogen, a virus or a microbe, or has a disease or condition,

(b) and if the individual in need thereof is found to be infected with the pathogen, the virus or microbe, or is determined to have or is diagnosed with the disease or condition, administering a drug or a treatment or agent for ameliorating or decreasing the symptoms of the disease, condition, pathogen virus or microbe, or administering a drug or a treatment to treat or ameliorate or decrease the symptoms of a condition, disease, infection or symptom caused by the pathogen, virus or microbe,

wherein optionally the disease or condition is cancer or a condition comprising inflammation.

In alternative embodiments, provided are kits or packages comprising:

(a) a product of manufacture, device, apparatus or system as provided herein;

(b) at least one set of buffer or media suitable for binding and washing;

(c) at least one set of detectable dyes or particles;

and optionally the kit further comprises detecting or secondary antibodies conjugated to detectable dyes,

and optionally further comprising a sample collection device or a target analyte processing device,

and optionally the target analyte processing device comprises a component or reagent for enrichment, extraction, purification, labeling, conjugation of the target analyte.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-B illustrate an exemplary design and device of an exemplary imaging system as provided herein for target analytes that are immobilized in a two-dimensional (2D) surface or a slide such as a microarray or biochip:

FIG. 1A is a schematic of an exemplary imaging device with external xyz dimensions in the range of between about 50 to 300 mm, noting that:

the sample is illuminated either from above and/or below with a light source parallel or at an angle with respect to the detection axis, and optionally the light source comprises a light-emitting diode (LED) or a laser diode;

optionally one filter or multiple filters is/are or comprises a spectral filter that can be used to further define the detection spectral window (for example, a short pass or band pass in one or multiple windows of between about 5 nm to 50 nm), and optionally the spectral filter is used to block excitation light, for example, long pass, single band or multi band in windows of between about 5 nm to about 200 nm;

a camera or equivalent capable of taking or capturing fluorescence images of the microarray, and optionally the camera or equivalent is capable of taking images of between about 0.2 to 200, or between about 1 to 100, megapixels, and optionally the camera or equivalent is fabricated as a charge-coupled device (CCD) or a Complementary Metal Oxide Semiconductor (CMOS) device, and optionally the camera or equivalent can take color or monochrome images), and the camera is operably coupled or connected to a lens or lens system (and optionally the lens is or comprises a plastic, glass or composite lens, and optionally the lens has a focal length of between about 1 to about 100 mm;

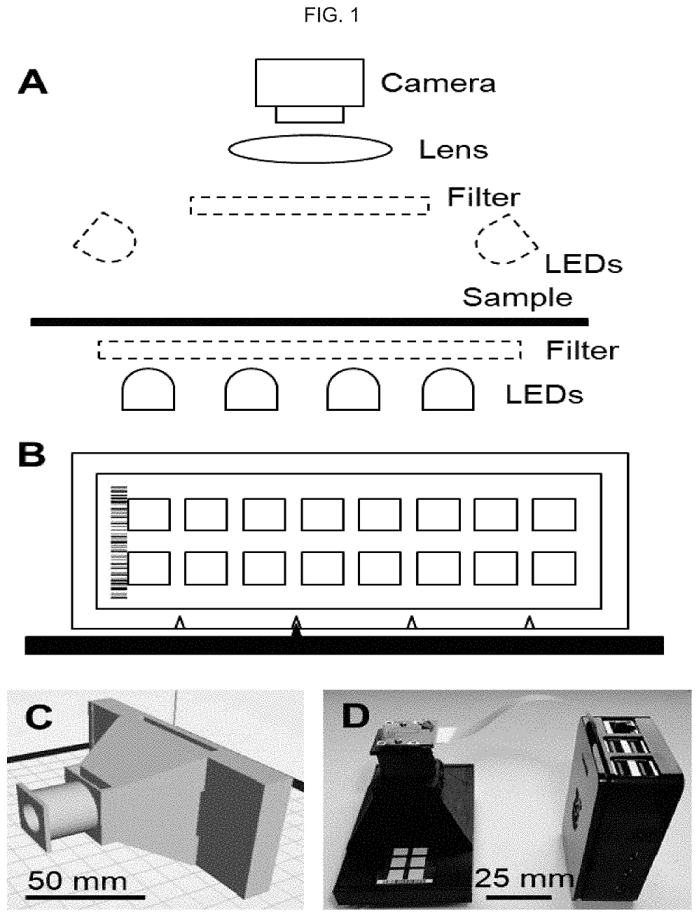

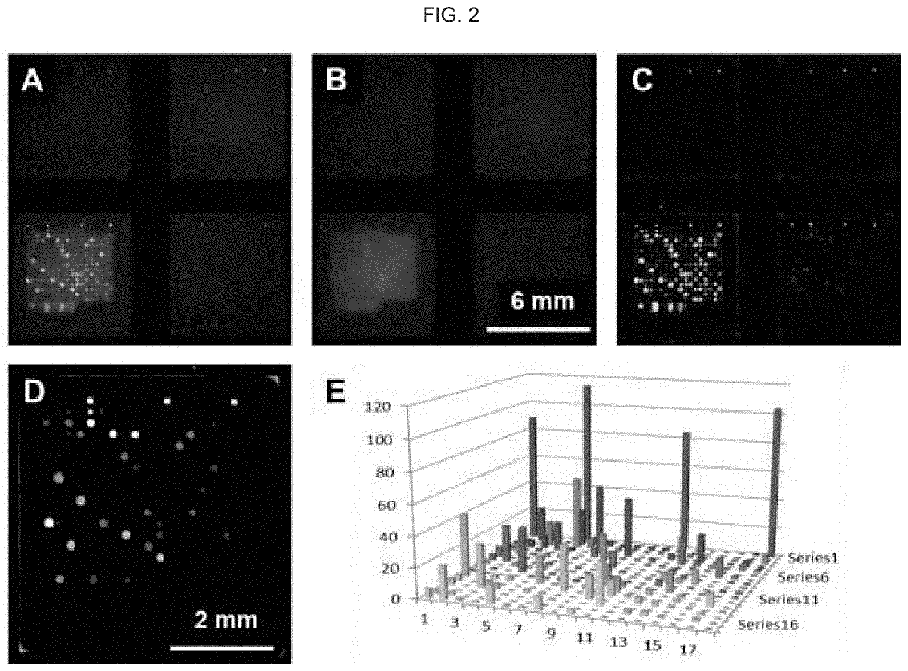

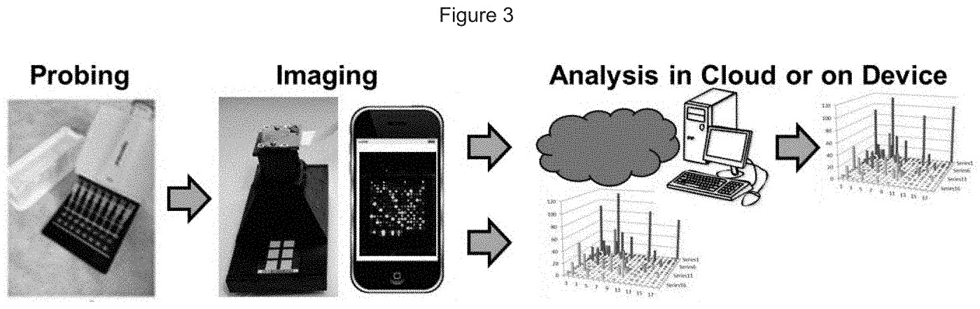

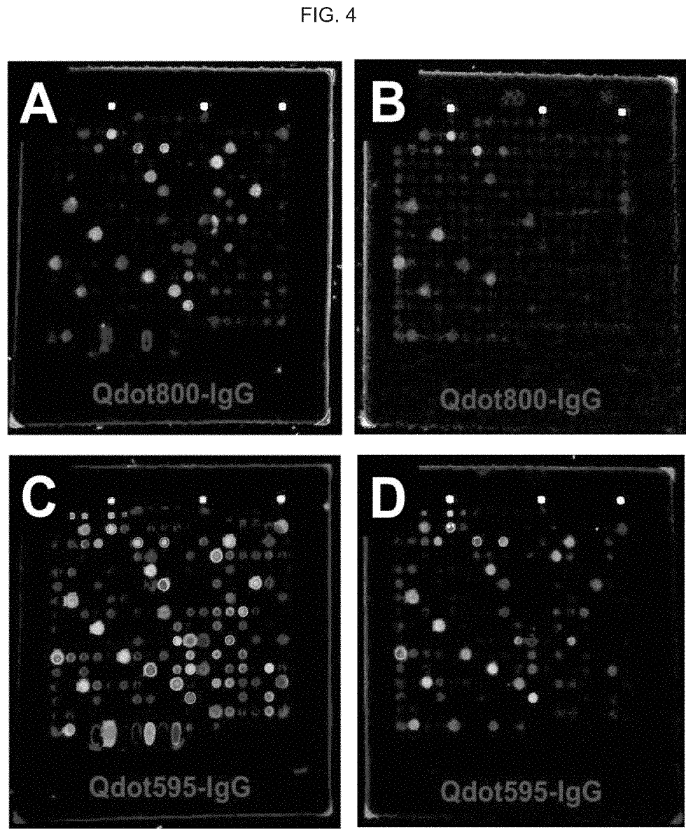

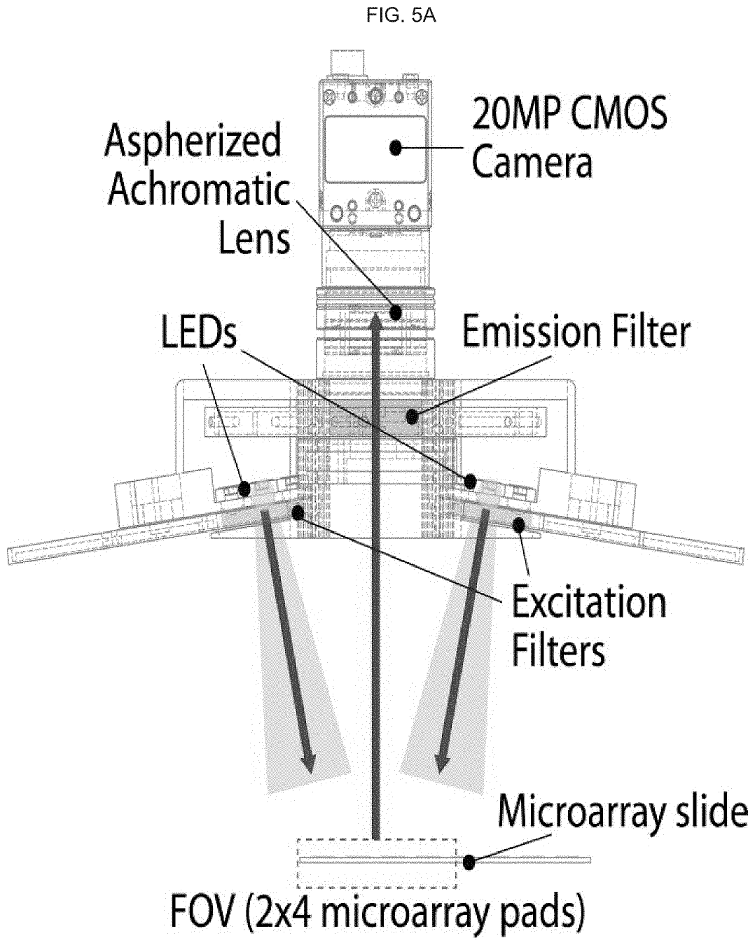

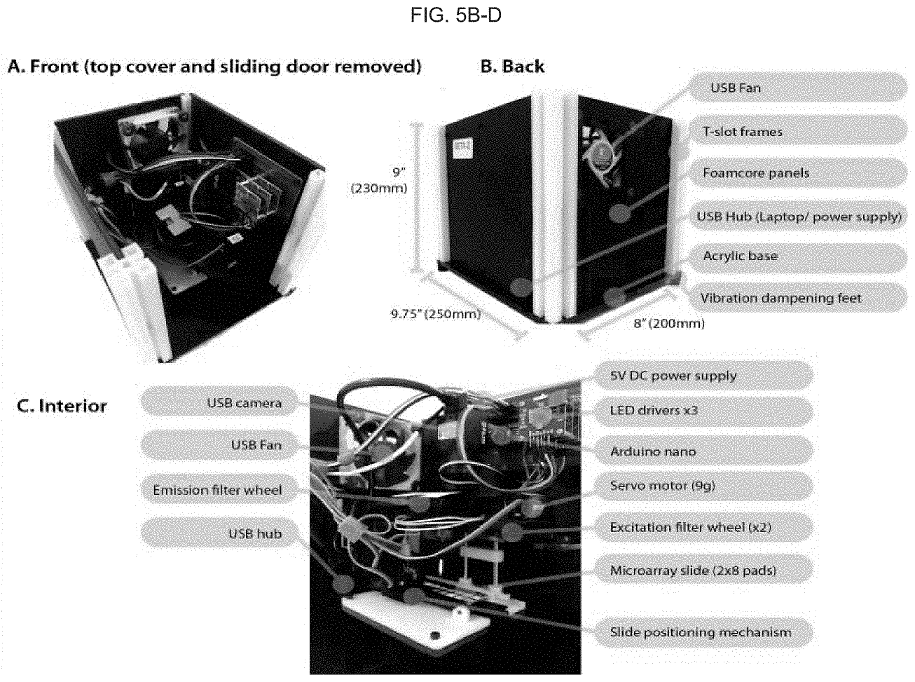

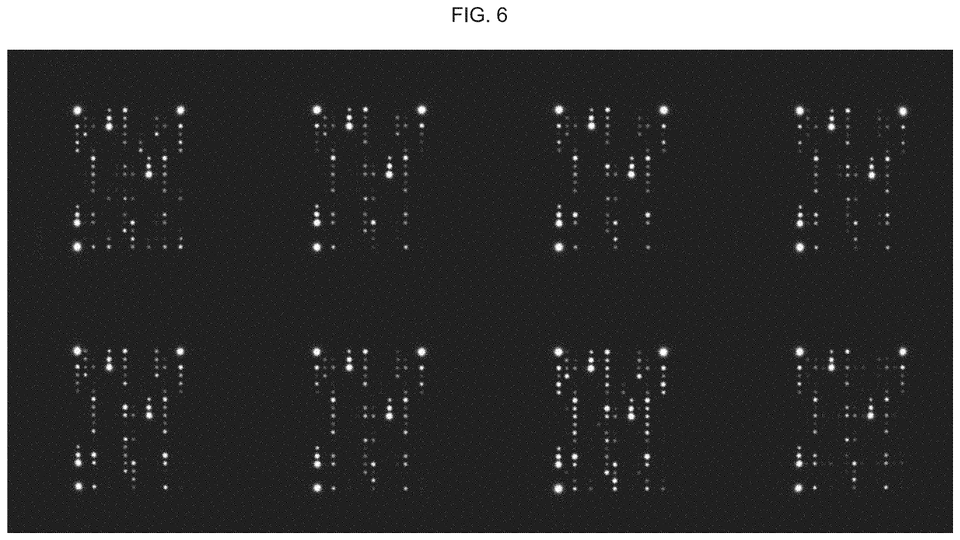

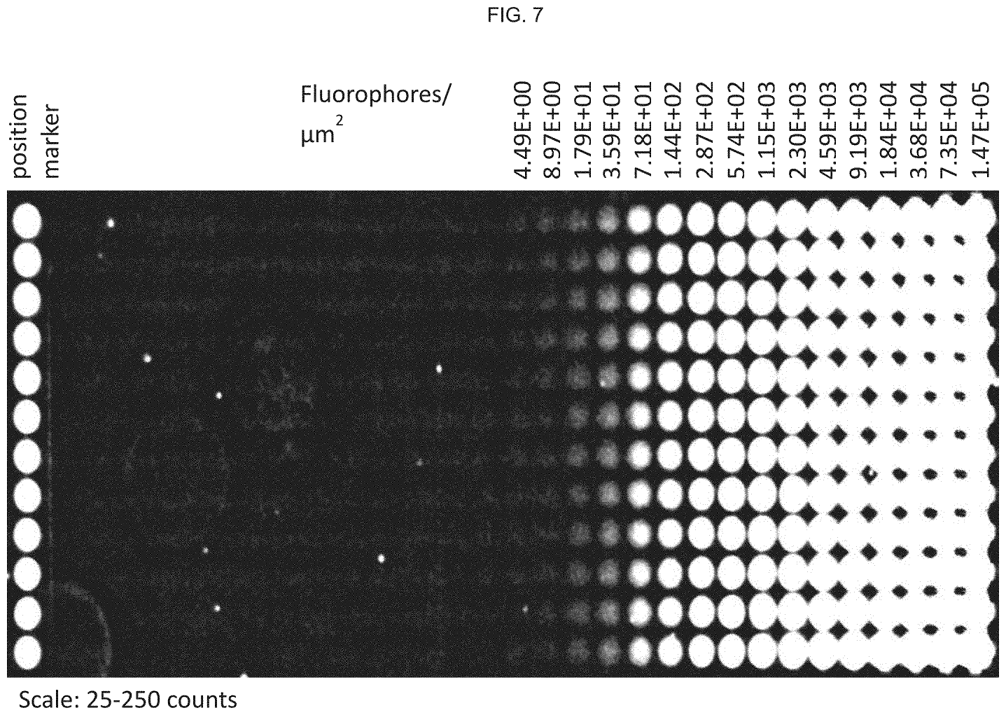

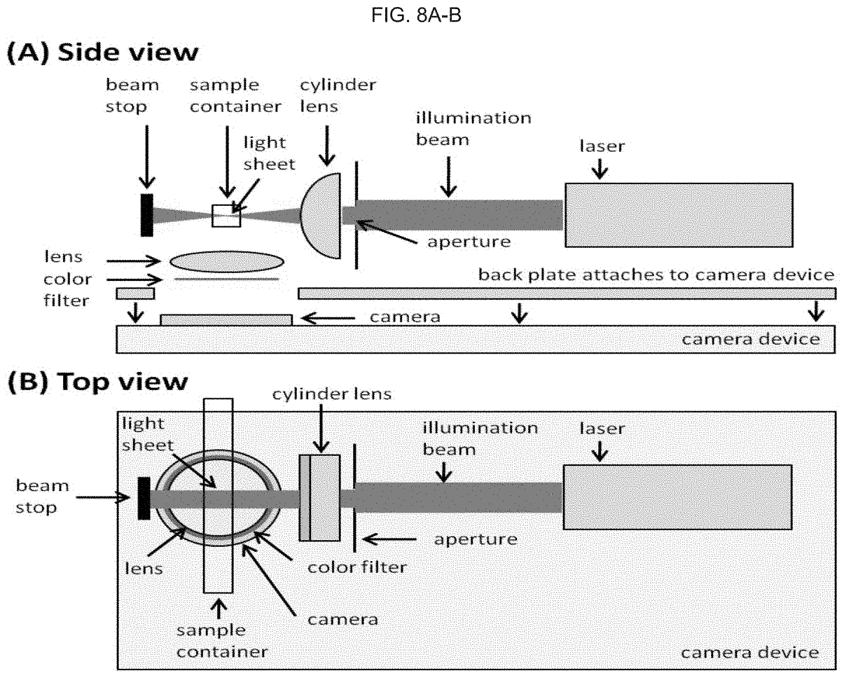

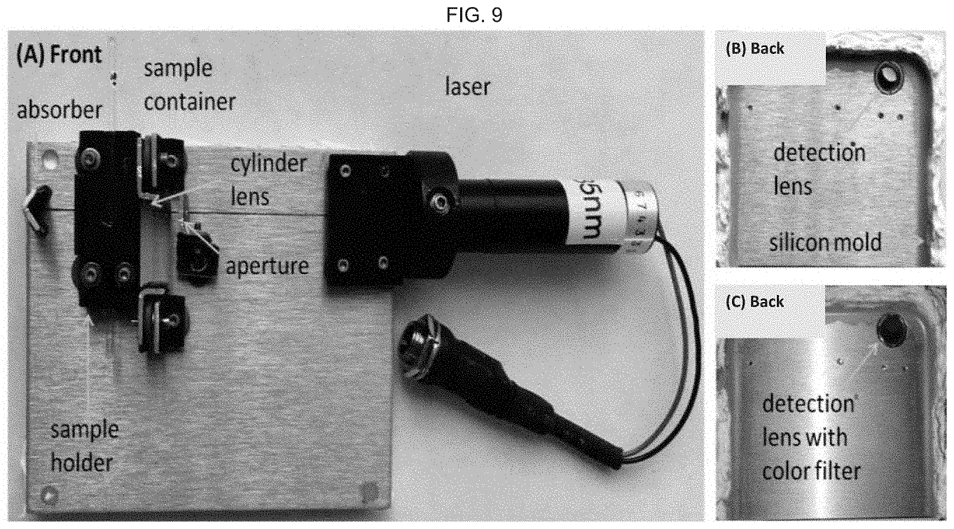

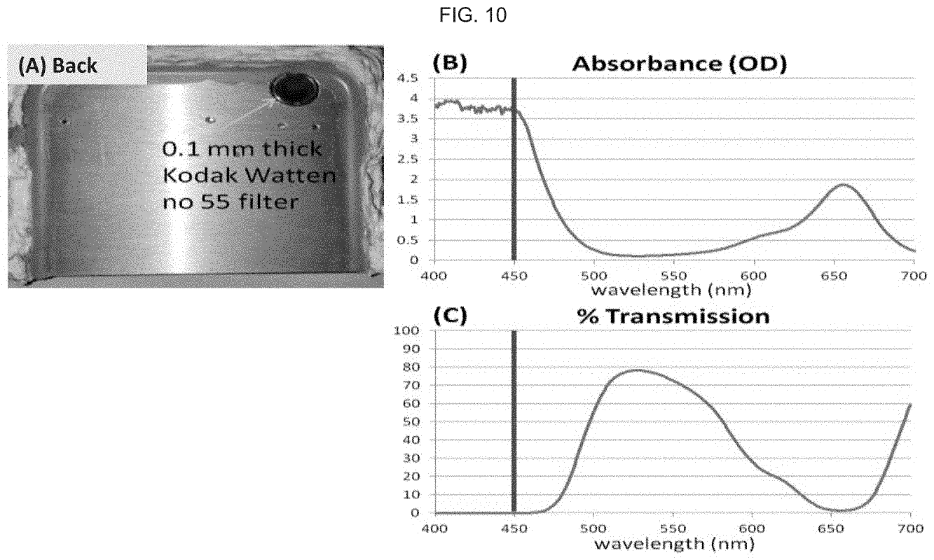

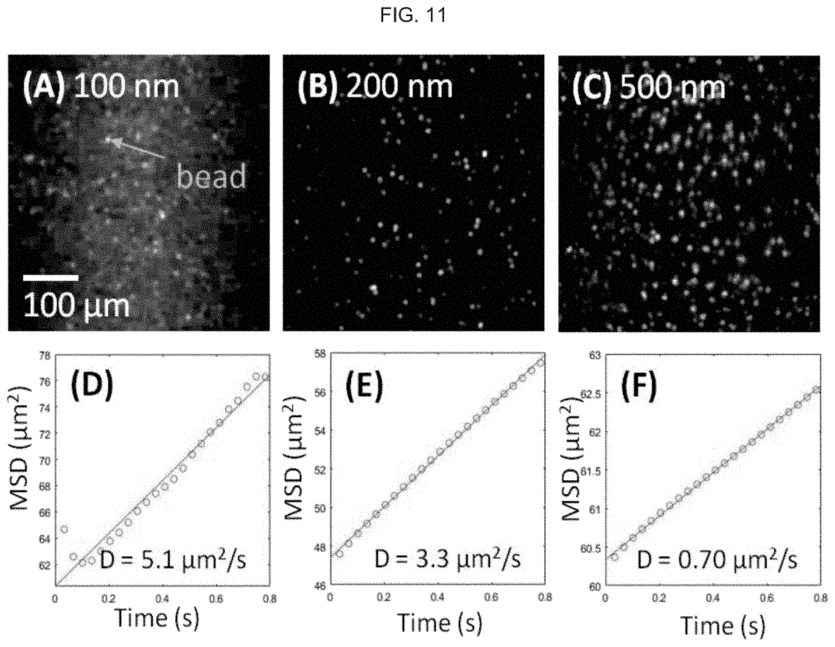

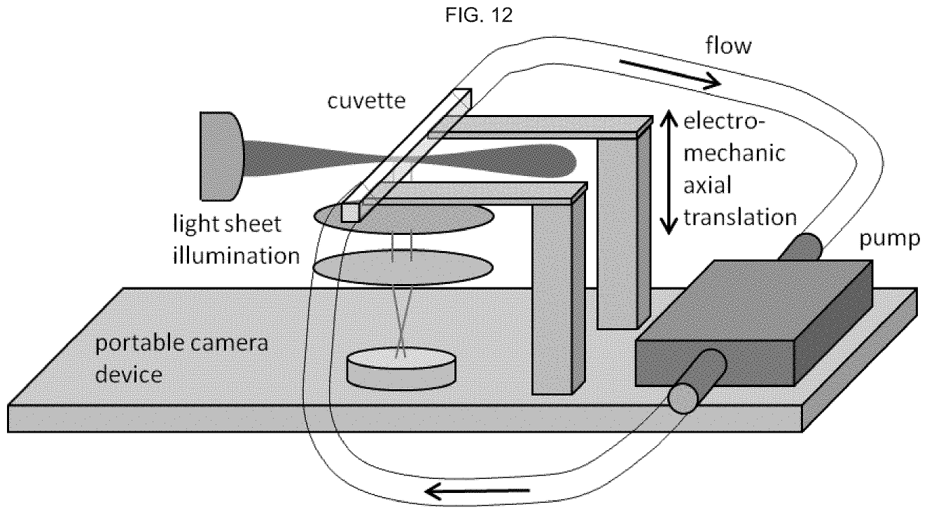

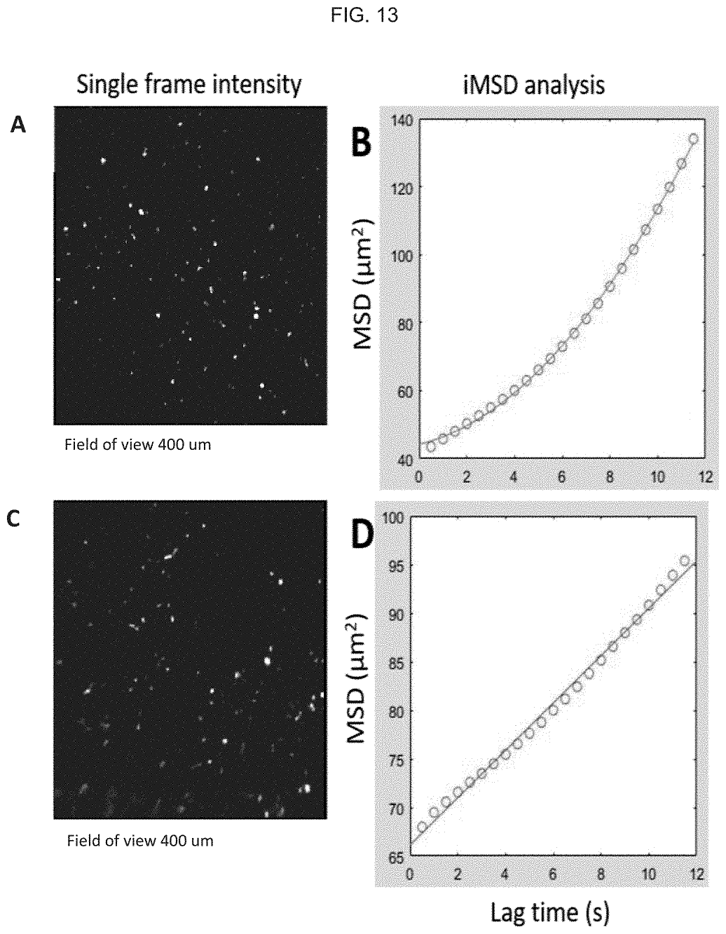

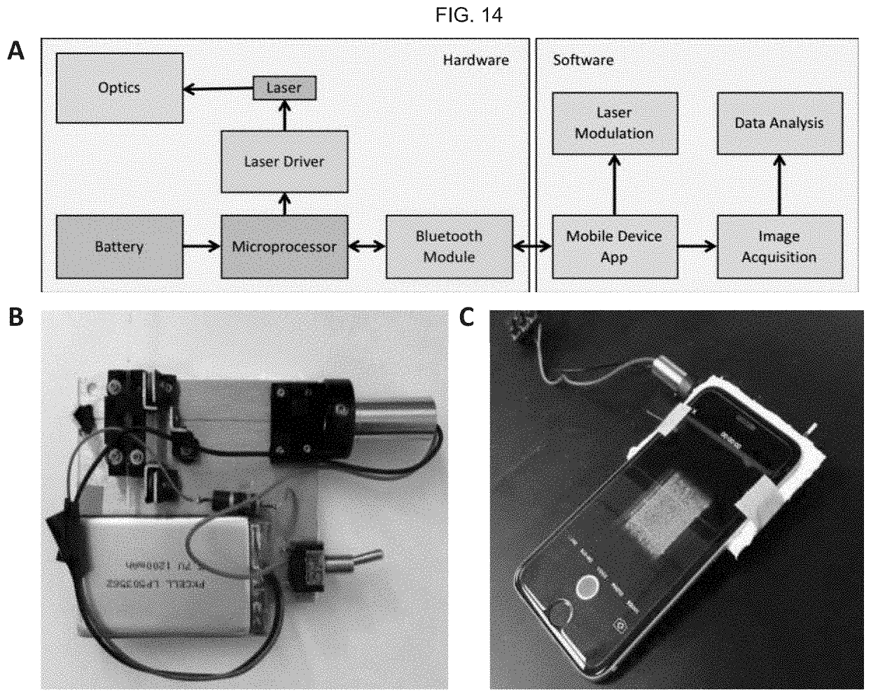

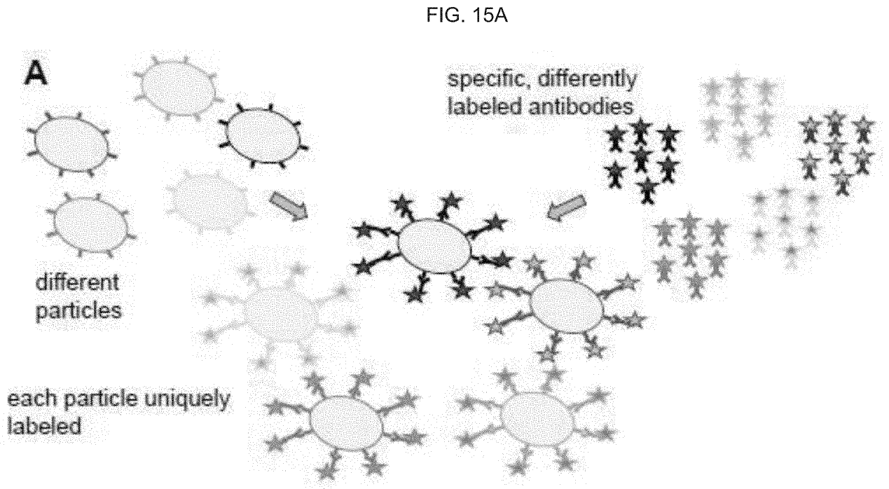

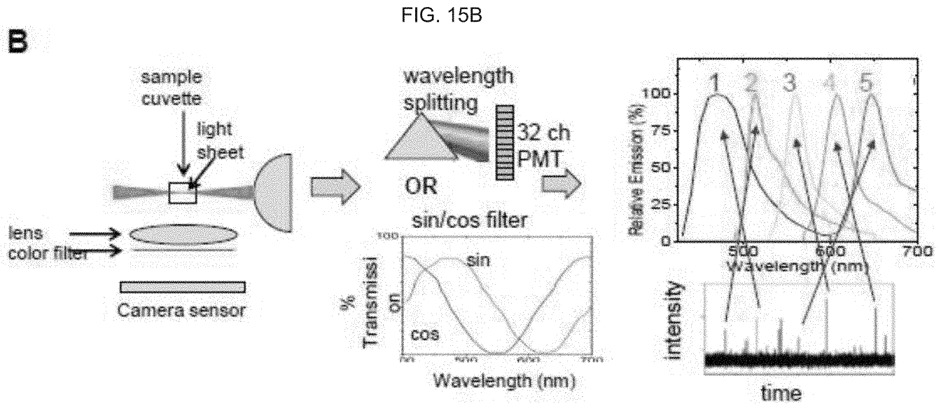

FIG. 1B is a schematic of an exemplary microarray format comprising a sample slide (for example, a 25 mm×75 mm sized slide of about 1 mm thickness) with several pads for microarray printing (for example, 2×8 pads, 6.5 mm×6.5 mm each pad) in a slide holder with alignment marks for consistent imaging of all pads (for example, 4 positions to take 4 images of 2×2 pads each),

and optionally the exemplary sample slide comprises a barcode attached or printed on the slide to allow for unambiguous identification of each slide, for example, for when the slide is scanned with a device camera or an external reader;

FIG. 1C is an image of an exemplary microarray imaging device, which can be a three dimensional (3D) Computer-Aided Design (CAD)/Computer-aided manufacturing (CAM) microarray imaging device, which optionally has the following features (from left to right): flange to adapt to the camera or mobile camera device, tube to contain optional camera lenses, inset for emission filter(s), housing to shield the imaging area from background and external light, slide holder to position the microarray in the camera field of view, inset for excitation filter(s), and mounting base for light source; and,

FIG. 1D is an image of an exemplary 3D printed and assembled microarray imaging device (left side image) with a camera or a sensor (for example, an OMNIVISION OV5647™ sensor) mounted, and an exemplary 25×75 mm microarray slide inserted into the imaging slot interfacing a single board computer (right side image, wherein optionally the computer is a RASPBERRY PI 4™)

FIG. 2A-E illustrate images, data and analyses of exemplary protein microarray imaging and analyses using imaging systems as provided herein:

FIG. 2A illustrates a raw image showing 4 pads of a 2×8 pad, 25×75 mm microarray slide with printed microarrays of a panel of antigens in a pattern of 17×18 dots in each pad, the slides were probed and developed with serum samples and with IgG-Biotin+Cy3 (top 2 pads) and IgG-Biotin+QD585 (bottom 2 pads), the sample was illuminated with 365-nm light specific to the excitation spectrum of QD585, and fluorescence was detected with a color camera (for example, an OMNIVISION OV5647™ sensor) coupled to a 570-nm long pass emission filter specific to the QD585 emission;

FIG. 2B illustrates an image where the low spatial frequency unspecific (non-specific) background was obtained by median-filtering the image shown in FIG. 2A with a kernel of 51×51 pixels;

FIG. 2C illustrates an image data after unspecific background removal shown in an overlay of green and red channels of the RGB color camera;

FIG. 2D illustrates a zoomed-in grayscale image of a single pad, the three bright spots on the top row are alignment markers to facilitate quantification; and,

FIG. 2E graphically illustrates quantification of fluorescence in all spots after alignment: for each spot in FIG. 2D the median intensity was calculated in an area of 10×10 pixels covering the spot spatial extension, the x and y axes of the graph correspond to the spot coordinate, the z axis is the median spot intensity as read from the camera chip.

FIG. 3 illustrates images of an exemplary pipeline of microarray-based analysis using a portable imager as provided herein: after probing with patient samples and staining with dye-labeled antibodies, the microarray slide is imaged by the exemplary portable imager, and images are acquired either with a mobile device with integrated camera or a dedicated camera interfaced by a computer, tablet, or smartphone, and fluorescence intensity values for each spot in each pad of a microarray slide are measured and calculated by on-device firmware/software, and by referencing this data to the known antigen layout of the microarray, probability values (p values) for the presence of antibodies against specific pathogens can be calculated, and this referencing step can be carried out either on the device itself or on a server after data upload.

FIG. 4A-D illustrates images acquired by an exemplary portable imaging devices as provided herein, the images are of antigen arrays labeled with quantum dot (QD) probes, and processed images showing single pads of a 2×8 pad, 25×75 mm microarray slide with printed microarrays of a panel of antigens in a pattern of 17×18 dots in each pad, where the slides have been probed and developed with serum samples and with IgG+QD800 (as in FIG. 4A and FIG. 4B, respectively) and IgG-Biotin+QD585 (as in FIG. 4C and FIG. 4D, respectively), and the sample was illuminated with 365-nm light specific to the excitation spectrum of QD585 and QD800, and fluorescence was detected with a color camera (for example, an OMNIVISION OV5647™ sensor) coupled to a 570-nm long pass emission filter specific to the QD585 and QD800 emission.

FIG. 5A-D illustrate an exemplary design and device of a microarray imaging system:

FIG. 5A illustrates a schematic of an exemplary imaging device with external xyz dimensions in the range of between about 50 to 300 mm, where the sample is illuminated from above with multiple light sources such as light-emitting diodes (LEDs) or laser diodes, and spectral filters are used to further define the spectral window (for example, band pass in multiple windows of between about 5 to 50 nm), and fluorescence images of the microarray are acquired with a camera (for example, a camera having between about 0.2 to 200 megapixels, or a CCD or CMOS, or a color or monochrome camera) coupled to a lens or system of lenses (optionally a lens comprising plastic, glass or composite, and optionally having a focal length of between about 1 mm to about 100 mm), and optionally emission filters block excitation light (multiple band pass filters in windows of between about 5 nm to about 200 nm), and the Field of View (FOV) can be a 2×4 microarray pad or pads;

FIG. 5B illustrates a schematic of an exemplary imaging device with door and top cover removed;

FIG. 5C illustrates a schematic of a back view of an exemplary imaging device with dimensions indicated (230 mm high, and 250 mm×200 mm), showing USB fan, T-slot frames, foamcore panels, USB hub, acrylic base and vibration dampening feet; and

FIG. 5D illustrates a schematic of an interior of an exemplary imaging device showing various internal components including: USB camera, USB fan, USB hub, slide positioning mechanism, emission filter wheel, excitation filter wheel, camera, microarray slide, and electronics to drive LEDs (for example, servo motor) and control the device.

FIG. 6 illustrates exemplary images of antigen arrays labeled with Quantum Dot probes (QD800), with images showing a single unstitched image of 2×4 pads taken of a 2×8 pad, 25×75×1 mm microarray slide with printed microarrays of a panel of antigens detecting coronavirus antibodies, where the slides were probed and developed with serum samples and with IgG plus QD800, and sample was illuminated with 470-nm light specific to the excitation spectrum of QD800, and fluorescence was detected with a monochrome camera (Sony IMX183™ sensor) coupled to a 780-nm long pass emission filter specific to the QD800 emission.

FIG. 7 illustrates an exemplary image of a calibration microarray with Cy5 spotted at different concentrations for each column as indicated in the figure; spot center-to-center distance was 350 μm; and the sample was illuminated with 620-nm light (red LED, cleaned with a 620/14 nm band pass filter) specific to the excitation spectrum of Cy5; and fluorescence was detected with a monochrome camera (Sony IMX183™ sensor, 5 second (s) exposure time) coupled to a 697/56-nm long pass emission filter specific to Cy5 emission, and a lens system with a numerical aperture of 0.02; and the image data indicate a lower limit of detection of less than (<) 20 fluorophores/μm² (signal-to-noise greater than (>) 3) for Cy5 with the filter, illumination and sensor configuration described; and this embodiment can be optimized by increasing illumination intensity, exposure time, numerical aperture.

FIG. 8A-B illustrate a schematic of an exemplary imaging system to detect (micro)particles in fluids in side view (FIG. 8A) and in top view (FIG. 8B); all components are mounted on a thin, rigid surface that acts as back plate for the mobile device enclosure (here, as an example, IPHONE (iPhone) 6S™); the collimated beam from a compact laser module (here, 635 nm or 445 nm) is directed onto an aperture with a diameter the size of the sample, which can be between about 0.5 mm to 50 mm (here, 1 mm); a cylinder optical element (here, f is 10 mm) is located immediately behind the aperture and focuses the light into a thin sheet, for example, can be between about 2 μm and 50 μm thickness (here, approximately 5 μm thickness) to illuminate a thin section of the sample; light is collected perpendicular to the illumination axis via a lens of short focal length, which can be between about 1 mm and 20 mm (in this example, 4.5 mm); and before entering the camera of the mobile device, the light can be filtered with an interchangeable color filter (here, KODAK WRATTEN NO 55™), for example, to alternate between scattered light and fluorescence light detection.

FIG. 9A-C illustrates an image of a photograph of the back plate of an exemplary (micro)particle detection device comprising an aluminum sheet of 1/16″ thickness, on the front, silicon sealant was used to match the dimensions and accommodate an IPHONE (iPhone) 6S™:

FIG. 9A illustrates an image of a front view of how all components are mounted on the exemplary device plate with 0-80 screws; to allow alignment, the individual components were suspended on 1/16″ thick rubber o rings: tightening of the screws compresses the rubber resulting in a translation range along the screw axis of approximately 1/32″; inserted into the device was an exemplary sample container, a square glass cuvette of 1 mm inner diameter, a wall thickness of 0.2 mm and a length of 50 mm, to focus the excitation beam into a sheet, a cylinder lens of focal length 10 mm and a dimension of 9 mm×14 mm was used, as light sources, a 635 nm and a 450 nm laser diode were used to illuminate the circular aperture of 1 mm diameter located immediately before the cylinder lens, the position of the cylinder lens was adjusted to focus the light in the center of the cuvette at the focal plane of the detection lens, an aspheric lens of 4.5 mm focal length (9 mm diameter); and, the sample holder can comprise a channel to accommodate the glass cuvette, an opening on the side to allow injection of the illumination beam and an opening on the bottom for light collection; an absorber behind the sample holder ensured that no excitation light leaves the mobile light sheet imaging platform;

FIG. 9B shows the back of the assembly shown in FIG. 9A with a silicon mold to connect to an IPHONE 6S™ camera with the detection lens; and

FIG. 9C shows the same view as in FIG. 9B but with a color filter mounted in front of the lens.

FIG. 10A illustrates an image of an exemplary mobile camera device adapter with an interchangeable color filter (here, KODAK WRATTEN NO 55™), a gelatin filter, which is placed between the detection lens and the camera objective, filter placement is shown in FIG. 10A; and

FIG. 10B-C graphically show: absorbance (FIG. 10B) and transmission properties (FIG. 10C) of the color filter of an exemplary device.

FIG. 11A-F illustrate images of an exemplary sample cuvette filled with yellow-green fluorescent latex particles of various sizes: 100 nm (FIG. 11A), 200 nm (FIG. 11B), 500 nm (FIG. 11C) suspended in water, placed in into the sample holder and subjected it to imaging; for illumination, 450 nm laser light was used and fluorescence light was collected through a KODAK WRATTEN NO 55™ filter via the mobile device camera (here: IPHONE (iPhone) 6S™), and videos of 1920×1080 pixels were recorded at 30 frames per second, and regions of 512×512 pixels were extracted and subjected to mean square displacement analyses, and the resulting diffusion coefficients are graphically shown in FIG. 11D-F: for the 100 nm (FIG. 11D), for the 200 nm (FIG. 11E), and for the 500 nm (FIG. 11F).

FIG. 12 illustrates an image of an exemplary portable camera device as provided herein having a tubing for liquid sample flow, the flow driven by a pump, also comprising an automated axial sample holder whose movement is controlled by a mechano-electric component, for example, a piezo actuator or voice coil actuator; and in combination with flowing the solution of interest through the cuvette, a volume of between about 0.1 ml to about 10 ml can be imaged in a few minutes with the ability to detect the presence of only a single particle; and potential applications are the detection of bacterial contamination of fluids like drinking water supplies, of medication such as fluids to be administered intravenously, or of fluid samples from patients such as blood or urine.

FIG. 13A-D illustrates the detection of Bacillus subtilis with an exemplary mobile phone light sheet imaging system as provided herein:

FIG. 13A illustrates a single frame intensity image of live B. subtilis as diluted in minimal medium and fluorescently stained with FM 4-64;

FIG. 13B graphically illustrates Mean Square Displacement (MSD) as a function of lag time obtained by image correlation analysis of 512 frames each, recorded at 29.9 frames per second (fps); a quadratic dependence of the MSD characteristic for active motion was found indicated by fitting the data with a parabola;

FIG. 13C illustrates a single frame intensity image of the same B. subtilis sample as shown in FIG. 13A subjected to heat shock treatment at 65° C. for 1 min; and

FIG. 13D graphically illustrates Mean Square Displacement (MSD) as a function of lag time as in FIG. 13B after heat shock, where the resulting MSD showed a linear dependence of the lag time that showed free diffusion without active motion indicated by a linear fit of the data.

FIG. 14A schematically illustrates a block diagram of hardware (left image) and software (right image) in an exemplary device assembly, in particular, and exemplary mobile camera sensing device; the block diagram shows exemplary hard- and software components: a light source illuminating the optics module is driven by a laser or LED driver that can optionally be controlled by a microprocessor, where the electronics can be powered by a battery, for example, the internal battery of a mobile device, or a power adapter; a bluetooth or wifi module can be used to communicate and control the imaging device; and software or firmware or mobile device app functionality includes, light source control or modulation, image acquisition and streaming, image data analysis and storage;

FIG. 14B illustrates an image of a photograph of a battery-powered exemplary mobile sensing device that uses a camera as illustrated in FIG. 14C, for example, an IPHONE (iPhone) 6S™, to generate, store, and process image data.

FIG. 15A-B illustrate exemplary multiplex image acquisition with a mobile camera device as provided herein:

FIG. 15A schematically illustrates several types of target particles or molecules that can be specifically labeled, for example with antibodies or antigens, with fluorophores of different excitation or emission properties such as emission spectra or lifetime; and,

FIG. 15B schematically illustrates an exemplary device with a slide or cuvette imager, where emission light can be resolved in the temporal or spectral domain; and for spectral detection, wavelength dispersion or splitting and a multichannel detector can be used; and optionally, sin/cos transmission filters can be used to spectrally resolve the emission signal and to separate and distinguish different components in the sample.

Like reference symbols in the various drawings indicate like elements.

### DETAILED DESCRIPTION

In alternative embodiments, provided are portable imaging systems and related kits, and methods for using them, for example, for the detection and analysis of analytes or biomarkers either: captured or immobilized on two-dimensional (2D) solid surfaces or slides, including microarrays or biochips; or, in solution or liquid phase by imaging 2D plane(s).

In alternative embodiments, provided are compositions, including products of manufacture and kits, and methods, for imaging, reading and/or analyzing proteins, antigens, or antibodies or biological particles based on 2D imaging detection devices such as microarrays, biochips and/or microbead assays. In alternative embodiments, provided are stationary or portable imaging systems to image, read, or analyze protein or antigen microarrays and particles (for example, cells, microbeads, nanoparticles) on surfaces and in fluids.

In alternative embodiments, imaging systems as provided herein are robust, inexpensive (for example, can cost between about $50.00 to $50,000.00), and can be deployed immediately at a point-of-care setting including those with minimal infrastructure including clinics, hospitals, pharmacies, drive through testing sites, customs and border checkpoints (for example, airports), and people's homes. In alternative embodiments, by uploading the resulting image data for cloud-based analysis with a smartphone or other mobile internet device, actionable results can be made available within minutes. In alternative embodiments, platforms as provided herein can make great impact to manage diseases and infections, contaminations, and control epidemics and pandemics such as COVID-19.

In alternative embodiments, products of manufacture as provide herein can be used to reliably read microarrays or other assay formats that have analytes captured, immobilized, or precipitated on solid surfaces (collectively called “slides” or otherwise 2D surfaces), and can: a) provide a large (for example, about 1 to 75 mm), uniform, undistorted field of view with high spatial resolution (for example, about 1 to 10 μm), b) enable sensor calibration to ensure a linear response in a quantitative or semi-quantitative fashion, c) provide the possibility to combine several spectral windows for illumination and detection to enable sample multiplexing, d) provide the possibility of light source and detector modulation to enable time-resolved detection of one or multiple targets, e) ensure consistent, homogeneous illumination and detection across all replicates of the device, f) interface to a data bank or computational facility to safely store and analyze patient data.

In alternative embodiments, products of manufacture as provide herein can be used to test a broad population for disease or infection screening, or screening during an epidemic/pandemic, in an inexpensive, high throughput manner. Alternative embodiments provide a portable imager or a reader that is: a) portable and of low cost, b) easy to use by non-experts with minimal training, c) feature low power consumption to allow battery powered operation, d) possible to manufacture on a large scale in a simple fashion.

In alternative embodiments, products of manufacture as provide herein can be used to detect analytes or particles in fluids in high throughput.

In alternative embodiments, provided are multiplexed, image analysis methods, apparatus and systems comprising, but not limited to, three major components: the hardware, the software or firmware, and the sample container (for example, a slide or a cuvette or a capillary or a flow cell); a block diagram of an exemplary device is shown in FIG. 14A, and the hardware can comprise the following components:

1) an optics module to generate homogeneous illumination of the sample, a lens or lens system to magnify the field of view of the camera, and one or multiple optical filters specific to the sample emission light (see for example, FIG. 1A, FIG. 5A).

2) optionally, the optics module can contain a cylindrical lens to generate a light sheet at the sample plane (see for example, FIG. 8A, B, 9A).

3) a camera for 2D image sensing that is either part of a mobile device or a stand-alone unit (see for example, FIG. 1D, 5A, 14C).

4) a laser or light-emitting diode (LED) light source to illuminate the sample in epi configuration or as a light sheet (see for example, FIG. 1A, 5A, 8, 9A).

5) a laser or LED driver to generate the laser or LED current (see for example, FIG. 5D).

6) optionally, a microprocessor to control the light source, detection unit, and optionally the sample position (see for example, FIG. 5D).

7) optionally, a Bluetooth, WiFi, Ethernet, or USB module for communication of the microprocessor with a mobile device or stationary computer (see for example, FIG. 5D).

8) optionally, a rechargeable battery or power adapter to supply power to all components (see for example, FIG. 14B).

The software or firmware can comprise:

1) a mobile device application that uses the Bluetooth or WiFi connection of the camera device to interface with the microprocessor of the imaging device (see for example, FIG. 3, FIG. 14C).

2) optionally, a wired or wireless connection to a stationary computer can be used to interface and control the sensing device (see for example, FIG. 1D, FIG. 14A).

3) functions of the application or software include, but are not limited to, after sample insertion the app will automatically choose the optimum illumination power, focus, exposure time and camera gain (self-calibration), for data acquisition the sample is moved in a manual or automated fashion (see for example, FIG. 5D), for multichannel acquisition the light source is switched and/or the detection filter or channel is switched (see for example, FIG. 5D).

4) the sample can be imaged with the mobile device camera optionally followed by automatic data analysis. Mobile device apps can be developed for multiple platforms (for example Apple iOS, Android).

5) analysis of data recorded with the image sensing device can be done by an application using the hardware resources of the mobile device (offline data analysis). The results can be communicated to a server/cloud service to be shared with others (online databank), see for example, FIG. 3.

In alternative embodiments, the sample is contained on or in a movable and adaptable sample container:

1) On a slide (2D functionalized surface, for example, printed microarray), or in a cuvette-like container (for example, to contain fluids). Slides can be standard coverglass slides (for example, 75 mm×25 mm×1 mm) that can have several pads or compartments (for example, nitrocellulose pads for protein printing), see for example, FIG. 1B, FIG. 2, FIG. 4, FIG. 6. As long as a flat surface is provided, slides can be from other materials including plastic (for example, polystyrene) and can be transparent or opaque; or

2) in a cuvette-like container including spectroscopy cuvettes, capillaries, flow cells, or microfluidic cells (see for example, FIG. 8, FIG. 9A, FIG. 12). The shape of the cuvette-like containers can be square, rectangular, or circular. The size of the cuvette-like containers can be between about 0.1 mm to about 10 mm in cross section and about 1 to about 100 mm in length. Optionally, the ends can be sealed after sample uptake or connected to a pump or other fluid flow or microfluidic device to flow sample during the container during imaging. For light sheet imaging, the sample container features two optically clear apertures perpendicular to each other, one for imaging and one for sheet illumination.

3) the sample container can be inserted into a sample holder that can be movable, optionally automated, in order to image a larger portion or the entire sample (see for example, FIG. 5D, FIG. 12).

In alternative embodiments, provided are camera sensing devices and multiplexed, microarray analysis methods, apparatus and systems comprising:

1) optical components for surface, slide and/or fluid imaging in a point-of-care device enclosure. In alternative embodiments, all components including illumination, detection optics, and the sample holder are integrated in a CAD/CAM model that can be manufactured on a large scale at low cost, for example, by 3D printing or injection molding. In alternative embodiments, a portable electronic device equipped with a camera-like sensor is used. In alternative embodiments, the components integrated into a device having external dimensions of between about 25 mm to 500 mm, and can include: a) light-emitting diode (LED) array or laser and LED or laser driver circuit, b) excitation filter, c) slide or cuvette holder, d) emission filter, e) a between about 0.2 to 200 megapixels camera (CMOS or CCD, color or monochrome) with external or integrated lens or lens system (see for example FIG. 1, FIG. 5, FIG. 7, and FIG. 14C). Illumination can be from the bottom, top, or at an angle (between about 0 to 90° to optical axis). Illumination can be in the form of epi-illumination or in the form of a light sheet or single plane injected into the sample at between about 85° to 95° (optionally between about 60° to 120°) to the detection axis. Products of manufacture as provide herein uniquely combine optical sectioning with a mobile camera device. Optical filters can be used to spectrally clean the excitation/emission light. If the emission band of the light source is sufficiently narrow (for example, a laser line, less than (<) 1 to 10 nm) an excitation filter is optional. In alternative embodiments, a color camera (for example, Red, Green, Blue (RGB) or RGBW (white added, an additive color system because when the three colors are combined in equal amounts, they form white) is used where the on-chip filter (for example, a color filter array (CFA), for a RGB Bayer pattern) can be used with or without an external emission filter.

In alternative embodiments, sample slides or cuvettes are inserted directly into the device in a dedicated slot that maintains the correct distance/position to the illumination (for example, between about 0 to 30 cm) and detectors (for example, between about 0 to 30 cm). Optionally, the sample is contained in a cassette or sled to adapt samples of different sizes (for example, between about 1 to 100 mm) to the same imaging device. A cassette or sled can also allow to quickly (for example, between 1 min to 15 min) image a larger number of samples (for example, between about 1 to 10 slides) at a time on a single device. For example, a sled as depicted in FIG. 1B, can hold between about 1 to 10 slides of between about 25×75 mm each and moved through the imaging device either manually, semi-automated, or fully automated. In manual operation, the operator moves the slide to the next imaging position and acquires the image. In semi-automated mode, the device software prompts the user to advance the slide to the next position and takes images automatically. In automated mode, there is an actuator (for example, DC or stepper motor, solenoid actuator, voice coil, piezo) integrated into an exemplary device (see for example FIG. 5D, FIG. 12) that advances the slide/sled/cassette in defined steps (for example, between about 1 mm to 100 mm).

In alternative embodiments, the slide holding mechanism ensures proper positioning to make sure that the individual pads of a slide can be indexed correctly (for example, see FIG. 1B, FIG. 2, FIG. 4, FIG. 6). This can be achieved by imprinting notches or other alignment marks at distances corresponding to 1 or multiple pads (for example, 5 mm to 50 mm) either on the slide holder/cassette or the slides themselves. Images of slides can be unambiguously identified by a barcode label located on each slide in the camera field of view.

An exemplary 3D CAD/CAM model of a single channel imager as provided herein is illustrated in FIG. 1C. After 3D printing of the model, all relevant optical components were inserted and attached. An exemplary photograph of the 3D-printed and assembled device together with a microarray slide inserted into the imaging slot and single board computer (RASPBERRY PI 4™) is shown in FIG. 1D. In the current device, for example, light from a 2×4 LED array mounted at the bottom is guided through 6-mm holes/channels to the individual pads of the microarray. In this example, the microarray comprises 2×8 pads of 6.5 mm×6.5 mm each. In this configuration, 2×4 pads can be imaged simultaneously with ample spatial resolution (10 μm sample pixel size) to resolve the individual dots (for example, between about 100 to 300 μm) of the microarray. Hence, the whole 2×8 array can be read by taking two images. For imaging, the device features a slide holder at the appropriate distance to the camera lens (in this example: 40 mm). After slide insertion into the slot, bump stops on each end ensure the correct position (pads 1 to 8 and pads 9 to 16) of the slide to reliably image all pads.

An exemplary schematic of a multichannel imager as provided herein is illustrated in FIG. 5A. After 3D printing of the individual components, all relevant optical components were inserted and attached. Exemplary photographs of the assembled device together with electronics and slide positioning mechanism are shown in FIG. 5B-D. In this exemplary device, light from pairs of blue (470 nm), green (520 nm) and red (620 nm) LEDs mounted at the top at a 7° angle to the detection axis are guided through emission filters interchangeable by a pair of filter wheels to illuminate the microarray. The microarray consisted of 2×8 pads of 6.5 mm×6.5 mm each. In this configuration, 2×4 pads can be imaged simultaneously with ample spatial resolution (10 μm sample pixel size) to resolve the individual dots (for example, between about 100 to 300 μm) of the microarray (see FIG. 6). The whole 2×8 array can be read by taking two images. For imaging, the device features a slide holder at the appropriate distance to the camera lens (in this example: 100 mm) that can automatically move the slide with a servo motor for fully automated imaging of the whole slide in multiple color channels.

2) Flexible Sample Illumination. In alternative embodiments, the sample slide is illuminated from the top, bottom, or side at an angle of between about 0 to 90° to the optical axis with one or multiple light sources in the visible (for example, between about 400 to 700 nm), near UV (for example, between about 250 to 400 nm), and/or near IR (for example, between about 700 to 1300 nm) range such as a light-emitting diode (LED) or a laser diode. Alternatively to epi-illumination, a light sheet can be generated with a cylindrical lens at the focal plane of the detection lens for fluorescence excitation. Optionally, broad band excitation light can be used and spectrally cleaned with optical filters (absorption or interference filters, short pass, long pass, or band pass with for example, between about 1 to 100 nm bandwidth). As an example, excitation in the microarray imaging prototype is realized with 2 LEDs mounted above the sample (FIG. 1 and FIG. 5). As another example, excitation in the fluid imaging prototype is realized with a laser diode (for example, between about 445 nm and 635 nm) mounted at a 90° angle with respect to the detection axis (FIG. 8 and FIG. 9), where a cylinder lens is used to create a thin (for example, between about 1 to 15 μm thickness) light sheet at the imaging plane. In alternative embodiments, multiple LEDs or lasers (for example, between about 1 to 100) with different (for example, between about 1 to 10) wavelengths can be used for multiplex applications. A bandpass filter is positioned between the LEDs and the sample to further narrow the LED emission spectrum. As an example, the slide imaging prototype can be equipped with the LEDs and filter combinations listed in Table 1. In alternative embodiments, other implementations include the use of multiband filters and illumination with laser diodes instead of LEDs. In alternative embodiments, multiple light sources can be integrated into the same device and switched independently with a controller and software to automate image acquisition in multiple color channels. In alternative embodiments, the LED or laser light sources can be modulated or pulsed to generate a time structured illumination. Time-resolved detection can then be used to increase sensitivity, reduce background, or to enable multiplex detection based on fluorescence or phosphorescence lifetime detection or spectrally resolved detection (FIG. 15).

TABLE 1
An exemplary set of fluorescence filter combinations
that can be used in the microarray imaging system.

| | LED | LED | Detection |
|---|---|---|---|
| Dye | wavelength | cleanup filter | filter |
| DyLight 405/QD | 365 nm | UG1 | GG420 |
| Alexa 488 | 470 nm | Magenta | Green |
| Alexa 594/Cy3 | 530 nm | Green | OG-570 |
| Alexa 647/Cy5 | 620 nm | BG-42 | R-660 |

3) Camera Detection. In alternative embodiments, images of slides or cuvettes can be acquired with a CMOS or CCD, color or monochrome camera with between about 0.2 to 200 megapixels coupled to a lens adjusted to a field of view of between about 1 to 75 mm. This can be a stand-alone camera interfaced by a computer or mobile device or a mobile device with integrated camera such as a smartphone. In alternative embodiments, spectral channels can be generated using a color camera and/or optical filters (optionally, long pass or band pass of between about 5 to 200 nm bandwidth). As an example (FIG. 1), one design uses a 5-megapixel color camera with an OMNIVISION OV5647™ sensor or equivalent, coupled to a 20 mm or 40 mm focal length lens for a field of view of 16 mm×32 mm, and a long pass filter specific to the desired fluorophore (here 570-nm long pass, other example illumination/filter combinations are shown in Table 1). The camera can be controlled by a single board computer (here RASPBERRY PI 4™) or interfaced with a smartphone or other mobile device (for example, tablet, laptop) to take images of the slide from the top. Another design (see FIG. 5) uses a 20 MP monochrome camera with a Sony IMX183 sensor, coupled to a lens system of 100 mm working distance for a field of view of 20 mm×37.5 mm, and multiple band pass filters specific to either QD585, QD655, or QD800. Another design (see for example FIG. 8-10) uses the integrated camera of an IPHONE (iPhone) 6S™, coupled to a 6 mm focal length lens, and a KODAK WRATTEN NO 55™ filter, to image a field of view of 1 mm×1 mm inside a square cuvette of 50 mm length and 0.1 mm internal cross section. In all prototypes, by exciting and detecting using different light paths, no dichroic mirror for separation of the fluorescence light is needed in the current prototypes. Exemplary microarray images acquired and processed with the current prototype are shown in FIG. 2, FIG. 4, FIG. 6, FIG. 7, microbead images are shown in FIG. 11. As a particle biosensing application, the image-based detection of bacteria (for example, B. subtilis) images is shown in FIG. 13A, FIG. 13C. In addition to detecting bacteria presence, particle tracking and analysis of the mean square displacement can distinguish between active motion and passive diffusion that can be used to distinguish live (FIG. 13B) and dead bacteria (FIG. 13D), for example, to enable antibiotic susceptibility testing. Optionally, several different species or types of target particles or molecules can be simultaneously detected by spectrally resolving the signal by means of several different detection filters or bands, or by dispersing the signal with a prism or grating into many (for example, between about 4 to 1024) spectral windows on the same camera chip, or by detection with filters with a response function in the shape of a linear ramp or sin/cos function (FIG. 15).

In alternative embodiments, the camera can be part of an integrated device such as a smartphone or a stand-alone camera module that is interfaced by a wired (USB, ethernet or dedicated) or wireless (Bluetooth or WiFi) connection.

In alternative embodiments, image acquisition comprises the following:
a) of the camera exposure time (for example, between about 1 μs to 10 s) and sensitivity setting (for example, between about ISO 64-ISO 128,000) to optimize the dynamic range to the fluorescence signal and ensure a linear response. Both over- and underexposure need to be avoided. This can be achieved by quickly (for example, between about 0.1 s to 5 s) taking a series of images (for example, between about 2 to 10 images) with different exposure/sensitivity settings (for example, about 0.1 ms, 1 ms, 10 ms, 100 ms, 1 s, ISO 100, ISO 200, ISO 400, ISO 800) of a reference sample with printed spots or particles of known dye concentrations or brightness distributed across the surface (for example, series of 0.3-0.001-fold dilutions of the same dye). The optimal exposure/sensitivity setting can then be determined by linear extrapolation of the calibration data.
b) Corrections of image distortions. To reliably quantify 2D images of surfaces and in solutions, it is important that the individual spots or particles appear at the correct sizes and distances. In alternative embodiments, cameras used in consumer electronics are often equipped with lenses of poor optical quality to reduce production cost. By measuring the distortions specific to a camera-lens configuration, resulting image distortions can be corrected. The most common type of distortion is spherical aberration often termed fisheye distortion. By taking a reference image of a well-defined target such as a checkerboard pattern (for example, 7×14 squares), the aberrations can be measured and corrected. Suitable algorithms are commonly available for machine vision applications (for example, Computer Vision (CV) Package for RASPBERRY PI™).
c) Background subtraction to eliminate spatial variations of the signal offset. To ensure a uniform low background, a high pass filtering (for example, 20 pixels cutoff) algorithm can be applied. By removing low spatial frequencies from the images, gradual variations in background can be removed (an example is shown in FIG. 2B, FIG. 2C).
d) Slide (for example, microarray pad) identification and alignment (an example is shown in FIG. 2D). To enable quantification of the dye concentration indicative of the protein concentration in all spots of a microarray, the intensity must be determined in each spot. For this purpose, microarray slides are typically printed with a distinct pattern of alignment marks on each pad. By calculating the correlation of the known alignment pattern with the image of each pad, the exact orientation and position of the array pattern can be determined. As the distances from the alignment marks to each spot and the size and distance of the spots are known (in this example: about 300 μm distance between spots), the average or median intensity can be accurately measured and quantified for each spot (illustrated as an example in FIG. 2E). In alternative embodiments, dyes, fluorophores or quantum dots with known concentration can be placed on the protein or particle detection assay for standardization, reference and calibration purposes. In alternative embodiments, positive, negative or internal controls (for example, saline, isotype controls) can be added to measure and correct for background or non-specific binding.
4) Data Processing. In alternative embodiments, firmware/software or algorithms deployed on the imaging device ensures acquisition of reproducible, quantifiable images of assay slides or cuvettes including, for example, printed protein microarrays, protein surface precipitates or (micro)particles or bacteria and other pathogens in solutions. In alternative embodiments, a Python programming language is used, and the following libraries: picamera, numpy, Image, scipy, and cv2. Python and packages, which are freely available at https://www.python.org/, also can be used. In alternative embodiments, the device ensures uniformity, linear response, background correction, sufficient spatial resolution, and separation into one or multiple spectral windows or temporal domains.

In alternative embodiments, an exemplary device as provided herein can:
a) acquire raw images with the exposure time adjusted to the brightness of the slide for maximum dynamic range (prototype python program commands: camera=PiCamera( ) camera.capture(output,‘rgb’)),
b) correct for spherical aberrations (prototype python program commands: map1,map2=cv2.fisheye.initUndistortRectifyMap(K,D,np.eye(3),K,DIM, cv2.CV_16SC2), img_cor=cv2.remap(img,map1,map2,interpolation=cv2.INTER_LINEAR, borderMode=cv2.BORDER_CONSTANT)) using parameters (K, D and DIM) obtained with a reference image of a checkerboard pattern (for example, 7×14 squares),
c) subtract background by using a high pass spatial filter algorithm (for example, 20 pixels cutoff, prototype python program commands: F1=fftpack.fft2((ch).astype(float), F2=fftpack.fftshift(F1), ch=fftpack.ifft2 (fftpack.ifftshift(F2)).real),
d) ensure automated pad alignment (prototype python program command: corr=signal.correlate2d(pad,markers)) utilizing reference markers on the slide (for example, 300 μm distance between spots). The slide layout can then be used to quantify antibody levels in each spot and which can be referenced back to the corresponding antigens. From those values, a probability (p value) for the presence of antibodies to a certain pathogen can be calculated. This analysis can be performed either on the imaging device itself or on a server after data upload (illustrated as an example in FIG. 3).

In alternative embodiments, an exemplary device as provided herein can use particle tracking or image correlation spectroscopy to quantify the movement and concentration of particles and molecules in solution. Example algorithms include:
a) Fluorescence correlation spectroscopy, where the signal in a single spot or camera pixel is recorded and autocorrelated as a function of time and the fluctuations caused by particles or molecules moving though the detection spot are analyzed to quantify the average number and average dwell time of those particles or molecules to inform on particle or molecule concentration and kinetics.
b) Image correlation spectroscopy, for example in the form of image mean square displacement analysis, where the signals between different spots or camera pixels are correlated as a function of time and distance between spots or camera pixels. As the probability to find a particle or molecule at or near the same location decreases over time if the particles can move, the particle or molecule kinetics can be quantified, as shown for microbeads in FIG. 11 and for bacteria in FIG. 13.
c) Single particle tracking, where the location of a particle is tracked by quantifying the center position of the particle intensity distribution in a series of images recorded as a function of time.

For image or image series acquisition and/or data analysis, a smartphone or tablet app can be used using a commercial development platform such as Appy Pie (www.appypie.com/); Alpha Anywhere (https://www.alphasoftware.com/) and Appery.io (https://appery.io/); or equivalents. This app could also utilize common APIs such as Google Drive. This way, the mobile app can sync the images to a cloud storage account where they can be stored and/or analyzed using custom routines and software as mentioned above or using third party software such as Mapix-CS (https://www.innopsys.com/en/lifesciences-products/microarrays/software/trial-versions). To ensure privacy, data must be protected by encryption with a personal account and password. In alternative embodiments, the patient or a health care professional can create an account with access code to review and store that microarray data. In alternative embodiments, the data management system must comply with the Health Information Portability and Accountability Act, as amended (HIPAA) (29 U.S. Code § 1181 et seq.) to protect information held by a covered entity that concerns health status, provision of healthcare or payment for healthcare that can be linked to an individual. With patient consent, de-identified anonymized data can be made available for clinical studies, statisticians, Institutional Review Boards (IRBs), and regulators. Clinical trial individual-level participant data (IPD) can be shared either as microdata (individual-level raw data) or through an online portal. These microdata can be one or more flat files or databases. The data can be analyzed only by qualified investigators (QIs) that are registered and have signed a HIPAA agreement. In alternative embodiments, when an online portal is used, the QI can access the data only through a remote computer interface, such that the raw data and all analyses shall reside on site. Data users do not download any microdata to their own local computers through this portal. Under this model, all actions can be audited.

In alternative embodiments, for example for clinical diagnostic purposes, target identification and classification can be accomplished by means of artificial intelligence (AI), machine learning, or deep learning of the recorded images or image series based on intensity, lifetime, spectral, size or morphology of the analyte. Types of algorithms include supervised learning, unsupervised learning, semi-supervised learning, and reinforcement learning.
a) In supervised learning, an algorithm is trained based on function approximation and the function that best describes the input data, i.e., for a given input X makes the best estimation of the output Y, is used. Example input data include image intensity pattern, lifetime, spectrum, pattern size or pattern geometry or morphology. Example output data include known positive or negative test results or known analyte concentration. To select the best function, training data containing the input/predictors as well as the correct output are used to model dependencies between the output and the input such that the output values for new data can be predicted based on those relationships learned from the training data sets. Common example algorithms include nearest neighbor, naive bayes, decision trees, linear regression, support vector machines, and neural networks.
b) In unsupervised learning, the computer is trained with unlabeled data and often used where it is unknown what parameter to look for in the data. Pattern detection and descriptive modeling algorithms try to mine the input data for rules or patterns, and summarize and group the data points to describe the data. For example, a set of imaging is given with known underlying condition such as a positive or negative diagnosis but where the parameter to look for such as a certain intensity or spectral or lifetime patterns are unknown are used for training. Common example algorithms include k-means clustering and association rules.
c) Semi-supervised learning, a combination of a) and b), is used when the data includes both known and unknown parameters. In the absence of labels in most of the observations but presence in a few observations, semi-supervised algorithms are used.
d) Reinforcement learning algorithms learn from the data iteratively until the full range of possible states is explored by repeated reinforcement feedback, i.e., a Markov Decision Process. Steps for reinforced learning include observation of the input state, use of a decision-making function to perform an action, reward or reinforcement from the result, the state-action pair information about the reward is stored and the process is repeated. Example algorithms include Q-learning, temporal difference, and deep adversarial networks.

In alternative embodiments, exemplary devices as provided herein use a general workflow or process of a surface or a slide (for example, microarray)-based analysis comprising: sample or specimen, sample collection, optionally sample processing, incubating the sample with capture slides, optionally a washing step, staining with dyes (or fluorophores, quantum dots) tethered with an affinity tag (for example, antibodies, oligonucleotides, peptides, proteins, aptamers, streptavidin, biotin, engineered tag or any combination of these molecules), optionally a washing step, followed by imaging and analysis with our portable system.

In alternative embodiments, exemplary devices as provided herein use a general workflow or process of microbead-based analysis comprising: specimen collection, optionally sample processing, incubating the sample with capture microbeads, capture antibodies or antigens, optionally a washing step, staining with dyes (or fluorophores, quantum dots) tethered with an affinity tag (for example, antibodies, oligonucleotides, peptides, proteins, aptamers, biotin, engineered tag or any combination of these molecules), optionally a washing step (for example magnetic separation based on magnetic microparticles), followed by imaging and analysis with our portable system.

In alternative embodiments, exemplary devices as provided herein use a general workflow or process of detecting and quantifying target analytes in solution. Example targets include viruses, bacteria, proteins, and other pathogens or molecular markers. Example human health related applications include the detection of bacterial infection, for example, presence of bacteria in urine, such as occurring in urinary tract infection (UTI), or sputum, such as occurring during upper respiratory tract infections (URI) as well as the presence of bacteria in potable water sources or food.

In alternative embodiments, exemplary devices as provided herein use a sample container with at least two optically clear apertures perpendicular to each other, one for imaging and one for sheet illumination. The sample is contained within such cuvette, capillary or flow cell. Optionally, the sample container is movable to image multiple regions of the sample. Liquid specimen together with assay solution are loaded into such sample cuvette or solid specimen are brought into solution before loading into the sample cuvette. Assay solution contains reagents and markers to generate image contrast to detect and quantify targets. Example reagents include antibodies, antigens, capture beads, nanoparticles, and fluorescent markers.

In alternative embodiments, exemplary devices as provided herein use a pump or other fluidic or microfluidic device is used to move the sample through the sample container or flow cell to increase the screening volume or to allow imaging of a time-resolved process or biochemical reaction or to process multiple samples in an automated fashion.

In alternative embodiments, exemplary devices as provided herein use an optical component arrangement for light sheet microscopy in a mobile device enclosure. All components including light sheet illumination, detection optics and the sample holder are attached to a mobile device enclosure. The device can be matched to the dimensions of any mobile device equipped with a camera.

In alternative embodiments, exemplary devices as provided herein record images or image series with an application or program using the hardware resources of the mobile device. The results can be analyzed on the device itself or communicated to a server or cloud service to be analyzed and stored and shared online.

In alternative embodiments, exemplary devices as provided herein use image processing to detect and quantify the presence of analytes, for example using pattern recognition, single particle localization and tracking, spectral and lifetime information, particle shape and size, and image fluctuation and image correlation spectroscopy-based analysis such as pair correlation and image mean square displacement analysis to measure particle number/concentration and velocity.

### Samples, Specimens and Sample Collection:

In alternative embodiments, specimens used to practice devices or methods as provided herein can be derived from human or animals (for example, a mouse, rat, or primate). Specimen types can include blood, serum, plasma, nasopharyngeal wash/aspirate or swab, nasal aspirate or swab, oropharyngeal (for example, throat swab), sputum, saliva, urine, tissues, and stool. These specimens can be collected using swabs for example in the cases of nasopharyngeal swabs, rectal swab and oropharyngeal swab. Sample processing steps, if needed, are well-established in the art for these specimens to extract, enrich, separate, isolate, purify, dilute or otherwise prepare target analytes prior to analysis.

In alternative embodiments, as an example, for COVID-19 or SARS-CoV-2 detection, specimens comprise blood, serum or plasma. Blood samples can be collected using apheresis collection, a syringe through venipuncture or a finger prick (or fingerstick) device. Plasma samples can be prepared from blood using centrifuge or a microfluidic device (for example, centrifugal microfluidic biochip, or CD microfluidics). For serum, blood is allowed to clot prior to separation by centrifugation. In alternative embodiments the samples are placed in appropriate tubes or container prior to analysis. Alternatively, upon collection using a finger prick, blood specimens can be collected and immediately flow through a device (for example, a microfluidic device) where be separated with a separate or automated device. (for example, automated flow through to the microarray or sample cuvette or capillary or flow cell). There are many automated or semi-automated devices or systems including cartridges that can perform blood draws, sample transport using flow-based mechanisms (for example, using capillary flow, lateral flow, pumps) and (in-line) sample processing such as plasma separation prior to analysis that we can use for this application (see, for example, Technology (Singap World Sci). 2018 6(2): 59-66). In alternative embodiments, as few as one drop of blood (or approximately 20 μl) can be sufficient for many clinical testing such as COVID-19 testing.

In alternative embodiments, the entire assay process including probing and imaging can be integrated. In alternative embodiments, for example, the process of probing the microarray or microbeads with sera including washing, incubation with the secondary antibody, incubation with substrate, and analysis of the results is automated. Robots and integration can be used in order to reduce the exposure risk of the operator and accelerate the process flow. In alternative embodiments, the microarray is printed on a rotating platform, or compact disc (CD), together with microfluidics (optionally, centrifugal microfluidic biochip or CD microfluidics) to drive, process and react the sample fluids and assay solutions within the system in a manual or automated fashion. In alternative embodiments, the assay solution (for example, containing capture beads and fluorescent markers), is pumped through a flow cell or capillary with microfluidics (optionally, centrifugal microfluidics) to drive, process and react the sample fluids and assay solutions within the system in a manual or automated fashion. The aforementioned imager can be part of such a system to include the imaging step. Instead of a sample holder/cassette, the imager can have a slot into which the CD can fit while still able to freely rotate. After the sample reaction, the sample can then be rotated into the appropriate position to take fluorescence images as described in previous embodiments.

In alternative embodiments, samples used to practice devices or methods as provided herein are derived from research and development processes such as proteins, cells, cell lines, cell lysates, tissue lysates, cell culture, biochemical reactions, drug and therapeutic manufacture process. In alternative embodiments, samples used to practice devices or methods as provided herein are drugs (or drug formulations) or therapeutics (or therapeutic formulations) including, for example, biologics, antibodies, and cell therapeutics. Sample processing steps, if needed, are well-established in the art for these samples to extract, enrich, separate, isolate, purify, dilute or otherwise prepare target analytes prior to analysis.

In alternative embodiments, samples used to practice devices or methods as provided herein are environmental samples, food, meat, water, beverage (for example, beer) for agriculture and environmental applications. Sample processing steps, if needed, are well-established in the art for these samples to extract, enrich, separate, isolate, purify, dilute or otherwise prepare target analytes prior to analysis.

### Target Analytes

target analyte can be a biomarker, protein, biologic, pharmaceutical, and a biological particle. A biomarker can be a DNA, RNA, protein, polypeptides, lipids, carbohydrates, polysaccharide, small molecules, metabolites. It is understood that multiple different analytes can be used and detected in a multiplex fashion to, for example, diagnose a disease.

Protein in this disclosure can be a peptide, epitope, polypeptide, antibody and antibody derivatives (for example, nanobodies, bispecific antibodies), antigen, cytokine, chemokine, enzyme, glycoprotein, hormone, signaling receptor, protein complex (for example, homodimer, heterodimer, or a multi-unit complex or structure), recombinant protein, synthetic protein, de novo designed protein, fusion protein, protein conjugate, modified protein (for example with polyethylene glycol (PEG), Fc domain, histidine, albumin, a dye) or a protein-based therapeutic. Particles in this disclosure comprise biological particles including, but not limited to, cells, mammalian cells, cancer cells, circulating tumor cells (CTC), mycoplasmas, platelets, immune cells, neural cells, engineered cells, fused cells, hybridoma, animal cells, plant cells, bacteria, viruses, fungi, parasites, pathogens, droplets, emulsions, therapeutic cells (for example, stem cells, T cells), a single molecule, a macromolecule such as protein and nucleic acid, a molecular product of a biochemical or enzymatic reaction, a RCA product, a RCA product labeled with dyes, or aggregates of biological molecules such as protein and nucleic acids. Particles in this disclosure also comprise microparticles, microspheres, microbeads, magnetic beads, nanoparticles (for example, quantum dots, qDot, silica nanoparticles, gold nanoparticles). The size of particles can range from 10 nm to 1,000 μm, and optionally from 100 nm to 100 μm. It is understood that particles can be in different shapes. Beads can be barcoded for downstream identification or target quantification (for example, as an internal reference) purposes based on size, shape, fluorescence spectral, lifetime and intensity properties by, for example, tuning composition of associated dyes, or using an oligonucleotide or a nucleic acid barcode.

In alternative embodiments, exemplary devices as provided herein, for example, microarrays or microbeads, can be used to analyze almost any type of molecules or particles in blood or other specimens, including immunoglobulins or antibodies (for example, IgG, IgM, IgA, IgD and IgE and their subclasses) that are produced as the immune system reacts to infections. In alternative embodiments, multiple isotype forms of antibodies including IgM, IgG and IgA will be analyzed using our technology for COVID-19 testing.

### Capture Target Analytes on Surfaces.

In alternative embodiments, products of manufacture as provided herein use any method or process of affinity, or binding, or capture-based mechanism to immobilize or capture target analytes from the sample. Many of these assay formats or surfaces (collectively called “slides”) or capture beads (functionalized micro- or nanoparticles) are established in the art such as microarrays, chips, arrays, microtiter plates (for example, for ELISA), (micro)wells, (micro)chambers, (micro)capillaries, lateral flow devices, membranes, (micro)beads, (micro)particles, nanobeads, nanoparticles, and microfluidics. In alternative embodiments, target analytes are captured on slides by covalent or non-covalent interactions. For instance, often used in the art is slides already immobilized with cognate binding molecules that capture target analytes utilizing biomolecular recognition such as antibody/antigen binding and nucleic acid hybridization. The said cognate binding or capture molecules typically are antibodies, oligonucleotides, peptides, proteins, aptamers, biotin, engineered tag or any combination of these molecules. Target analytes can also be captured on slides by other forms or interactions such as ionic, H-bonding, hydrophobic, electrostatic or metal/chelate interactions, centrifugation force, gravity or buoyancy. In alternative embodiments, target analytes can be first captured by particles (for example, microbeads) and then deposited on slides, surfaces, (micro)wells, (micro)chambers, in capillaries, cuvettes, flow cells, or encapsulated in droplets. Samples can be directly added and incubated in cuvettes, on slides or pass through the slides using a flow-cell, microfluidic device, tube, channel, or a capillary. In alternative embodiments, samples (for example, urine) can pass through a membrane or filter to collect, capture or enrich target analytes (for example, bacteria) and subsequently targets can be stained on the said membrane or filter which can be directly imaged with disclosed imagers herein. In alternative embodiments, for our fluid imaging system, samples can be directly interrogated in a container such as a cuvette or using a flow-through system such as a flow-cell, microfluidic device, tube, channel, or a capillary. In alternative embodiments, slides are manufactured with different dimensions, morphology, topography and structures to, for example, modulate binding interactions or flow dynamics.

All these “slides”, “cuvettes” or flow-through devices mentioned above including microarrays, chips, arrays, microtiter plates (for example, for ELISA), (micro)wells, (micro)chambers, (micro)capillaries, lateral flow devices, membranes, (micro)beads, (micro)particles, nanobeads, nanoparticles, microfluidics, flow-cells, tubes, and capillaries can be imaged using imagers provided herein. Here we use microarrays and square cuvettes as an example to illustrate the assay process; in alternative embodiments, any form of slide, cuvette, capillaries or flow-through systems can be used to practice products of manufacture and methods as provided herein.

In alternative embodiments, products of manufacture as provided herein use any method or process of affinity microarray preparation. In alternative embodiments, affinity moieties including nucleic acids, proteins, antigens, pathogens, carbohydrates and other affinity tags are used, and can be printed, adsorbed, deposited or chemically conjugated on an exemplary microarray format for bioanalysis, which can all be read and analyzed using an imaging system as provided herein. In alternative embodiments, microarray comprise nitrocellulose membrane, glass, silicon, plastic or a polymer substrate.

In alternative embodiments, products of manufacture as provided herein comprise or are fabricated as coronavirus antigen microarrays that can detect antibodies against a panel of antigens (see, for example, FIG. 4, FIG. 6). In alternative embodiments, coronavirus antibodies can be printed on microarrays to detect viruses or viral antigens. In alternative embodiments, coronavirus antigens used in products of manufacture as provided herein comprise one or multiple types of antigens, including for example coronavirus spike protein (S), nucleocapsid protein (NP), membrane (M) protein, envelope (E) proteins or any other viral constituents such as RNA and proteases. It should also be understood that each protein antigen may come as many different variants and can be used alone or in combination on the microarray.

In alternative embodiments, products of manufacture as provided herein comprise or are fabricated to use coronavirus S proteins comprising different receptor-binding domains (RBD) including S1 and S2 domains, which can be separately printed or used in combination as whole proteins (S1+S2) on microarrays. In alternative embodiments, products of manufacture as provided herein comprise or are fabricated to use antigens for SARS-CoV-2, and/or antigens for other coronaviruses such as SARS-CoV, MERS-CoV, and/or common cold coronaviruses (for example HKU1, OC43, NL63, 229E) as well as other viruses or pathogens (for example, influenza, adenovirus, MPV, PIV, RSV and HIV).

In alternative embodiments, products of manufacture as provided herein print antigens in multiple spots on a slide or equivalent surface to serve as internal replicates to increase testing accuracy. In alternative embodiments, microarray preparation is modular, and new antigen variants of viruses can be included as they continue to evolve. These antigens can be derived and prepared from viruses or expressed in host cells (for example, HEK-293 cells) through recombinant technologies. Many of them are also available at commercial vendors (for example, Sino Biological). The antigens can be printed onto microarrays including nitrocellulose-coated slides using a microarray printer. These slides can be stored in a desiccator for a long period of time or shipped without losing biological functions.

### Target Probing and Luminophores

Target analytes, captured on a surface or otherwise in liquid or solution, are typically probed or detected with luminescent probes (for example, fluorescence, phosphorescence, chemiluminescence). For captured target, for example, a sandwich assay can be used involving probing the captured target with detection antibodies tagged with a luminophore (or molecules that can generate light through a biochemical reaction such as fluorogenic enzymes including, for example, horseradish peroxidase, alkaline phosphatase, β-galactosidase or luciferases) or optionally further labeled with secondary antibodies tagged with a luminophore (for example, FITC-anti-mouse IgG). In alternative embodiments, any affinity binding partners can be used, for example, (strept)avidin and biotin linkages can be used to label target with luminophores. In alternative embodiments, secondary probes can be antibody-oligonucleotide conjugates or oligonucleotide probes for multiplexing and barcoding purposes. In alternative embodiments, proximity ligation assays can be used for protein detection. In alternative embodiments, biomolecules are modified using standard bioconjugation techniques to add functional moieties such as biotin (through “biotinylation”), dyes, or purification tags (for example, Fc, histidine) to enable different purposes. In alternative embodiments, signal amplification processes established in the art such as rolling circle amplification (RCA) and tyramide signal amplification (TSA) can be conducted to amplify signals.

In alternative embodiments, capture particles (for example, microbeads) can be used with a surface chemistry that enables binding and subsequent labeling of targets with affinity luminophores prior to analysis. In alternative embodiments, the particle of interest (for example, cells, bacteria, yeast cells, viruses, protein or nucleic acid aggregates) can be marked and detected directly with luminescent probes. Labeled particle targets can be detected after precipitation from the liquid phase to a surface or directly in the liquid or solution phase using light sheet illumination. In alternative embodiments, washing steps are involved to remove unbound molecules or probes. In other embodiments, assays can be performed in a “homogenous” fashion without washing steps (for example, Förster resonance energy transfer (FRET), bioluminescence resonance energy transfer (BRET), time-resolved FRET or BRET, and molecular beacon designs).

In alternative embodiments, luminophores can be any light (for example, fluorescence, phosphorescence, chemiluminescence) emitting moieties. As an example, detectable dyes that can be used in alternative embodiments comprise: DyLight 405, FAM, FITC, Cy2, Cy3, Cy5, Alexa488, Alexa594, Alexa647, qdots (for example, QD585, QD655, QD800), polymethine dyes, luminescent lanthanide complexes, Europium, Rubrene, upconversion nanoparticles or dyes, and ultrabright fluorescent labels comprise scaffold materials associated with a plurality of dyes (for example AIE beads from Luminicell). In alternative embodiments, any light-emitting moiety can be used herein including, but not limited to, the BODIPY series, the Alexa series, the ATTO series, PE, Coumarin, PerCP, TRITC, Texas Red, and APC. In alternative embodiments, fluorescent protein (for example, Green Fluorescent Protein (GFP), Cyan Fluorescent Protein (CFP), Yellow Fluorescent Protein (YFP), and a Red Fluorescent Protein (RFP)), or split fluorescent proteins, split luciferases and other split enzymes can be used herein to emit light. In alternative embodiments, luminophores are used in combination in a bioassay such as in FRET or BRET or Time-resolved FRET or BRET.

In alternative embodiments, biological particles or cells can be directly labeled using a dye or luminophore conjugated to an affinity tag described above (for example antibody) or particles (for example beads, nanoparticles emulsions, or vesicles). For example, an antibody-dye can bind to a particular molecule on the cell membrane or inside the cell. In alternative embodiments, biological particles or cells can be labeled by other means including, for example, metabolic dyes (for example, resazurin), live or dead cell stains or viability dyes (for example, 7AAD, DAPI, PI), apoptotic marker (for example, annexin V), DNA intercalating dyes (DAPI, 3,5-difluoro-4-hydroxybenzylidene imidazolinone (DFHBI), thiazole orange, Propidium Iodide, SYTO 9, and SYTOX or their derivatives and variants). In alternative embodiments, biological particles or cells can be marked with engineered reporters (for example, fluorescent proteins expressed in mammalian cells, or phage mediated bacterial expressing fluorescent proteins).

In alternative embodiments, antigen microarray slides provided herein as an example are probed with specimens as described above (for example, human sera, plasma, or whole blood) in appropriate media and preparations (for example, dilution) for a period of time ranging from between about 5 min to about overnight at between about room temperature (RT) to 4° C., or at RT or 4° C. In alternative embodiments, a washing step is performed prior to labeling with (for example, secondary) antibodies, for example, to human IgA and IgG, for example, conjugated to dyes, fluorophores or quantum dots or equivalents for another period of time, for example, ranging from between about 5 min to 6 hours, or about 2 to 3 hours, for example, at room temperature (RT). In alternative embodiments, a final washing step is performed with appropriate buffer to remove any unbound moieties. In alternative embodiments this entire process, or one or more steps of the process, are automated. The final sides can be analyzed immediately or dried and stored until analysis using our disclosed imaging systems as described above.

### Microarray-Based Clinical Diagnostics and Research Tool for Therapeutic and Vaccine Development

Methods, apparatus and systems as provided herein can broadly enable in vitro diagnostics (IVD) or companion diagnostics (CDx) using microarrays including for example DNA or RNA arrays (or gene chip) and protein arrays. As summarized by Wu et al. (BioTechniques 39:577-582), gene chips can be used for a variety of nucleic acid analysis including, for example gene expression, chip-based sequencing, single nucleotide polymorphism (SNP) analysis, genotyping different polymorphisms, sequence variation. An example of protein microarray is antibody microarray which can be used to profile for example disease markers, cytokines, and signaling molecules in a broad range of research (for example tissue culture, cells, cell or tissue lysates and biological manufacture processes) and clinical disease settings (for example cancer or infectious diseases).

In alternative embodiment, microarrays as provided herein and as used in devices as provided herein can be read by imagers disclosed herein and assayed using methods, apparatus and systems as provided herein, instead of using more complicated and expensive microarray scanners in the art. The methods, apparatus and systems as provided herein can therefore find a broad range of applications in therapeutic and vaccine development and in clinical diagnostics.

In alternative embodiments, microarrays as provided herein are broadly applicable to any 2D surface-based assay platforms including for example chips, glass slides, cover slides, microscope slides, microtiter plates (for example, for ELISA), (micro)wells, (micro)chambers, (micro)capillaries, lateral flow devices, membranes, (micro)beads, (micro)particles, nanobeads, nanoparticles, and microfluidics.

### Protein Detection

In alternative embodiments, provided are products of manufacture, methods and kits for detecting and analyzing proteins including, for example, cytokines, antibodies, and antigens in biological or clinical samples. In alternative embodiments, any established protein detection chemistries and assays or immunoassays can be used to practice products of manufacture, methods and kits as provided herein (for example imaging systems and methods as provided herein), for example, as described in sections “Capture target analytes on surfaces” and “Target probing and luminophores”. In alternative embodiments, any protein assay or immunoassay can be used to practice products of manufacture, methods and kits as provided herein. For illustrative purposes, we describe herein general practices using a bead-based sandwich format for protein detection. In alternative embodiments, target proteins in the sample are captured on cognate antibody-immobilized microbeads. The captured proteins are then probed in a sandwich assay using detection antibodies (or together with secondary antibodies) tagged with a fluorophore, Qdot, or ultrabright nanoparticles (for example AIE beads, or biocompatible organic fluorescent nanoparticles (AIEDots), from LUMINICELL™). In alternative embodiments, the captured protein can initiate an enzymatic reaction such as rolling circle amplification (RCA) to amplify signal. Optionally, bead washing steps are performed in between these steps. Subsequently, the microbeads are then deposited on slides, surfaces, (micro)wells, or (micro)chambers or suspended (or flowed through) in capillaries, cuvettes, or flow cells, which can be imaged and analyzed with our disclosed imaging systems herein (for example FIG. 1, FIG. 5, FIG. 8, FIG. 12, FIG. 14).

In alternative embodiments, provided are products of manufacture, methods and kits for detecting and analyzing single or individual proteins (for example, for “digital” protein detection). In alternative embodiments, digital protein detection is enabled by imaging individual protein molecules tagged with superbright nanoparticles (for example AIE beads, or biocompatible organic fluorescent nanoparticles (AIEDots), from LUMINICELL™) or long rolling circle amplification (RCA) products.

### Products of Manufacture and Kits

In alternative embodiments, provided are products of manufacture and kits for practicing methods as provided herein; and optionally, products of manufacture and kits can further comprise instructions for practicing methods as provided herein. In alternative embodiments, provided to practice our disclosed slide imaging and analysis systems are a blood drawing device, a blood processing device, printed microarrays, staining reagents including antibodies, antibodies tethered with dyes, fluorophores or quantum dots, control samples, and incubation, blocking and washing buffers. In alternative embodiments, provided to practice our disclosed slide imaging and analysis systems are a blood drawing device, a blood processing device, capture (micro)beads, staining reagents including antibodies, antibodies tethered with dyes, fluorophores or quantum dots, control samples, and incubation, blocking and washing buffers.

### Exemplary Applications

In alternative embodiments, methods, apparatus and systems as provided herein enable point-of-care diagnosis, clinical diagnosis, immunity analysis, research, epidemiological surveillance, and/or therapeutic and/or vaccine development, patient stratification, or measuring therapeutic outcomes in a broad disease settings including for example cancer, inflammation, pain, autoimmune diseases, infections (for example, bloodstream infections, urinary tract infections, antibiotic susceptibility testing), cardiovascular diseases, central nervous system (CNS) disease. For instance, methods, apparatus and systems as provided herein can have broad utility and implications in addressing, for example, COVID-19 pandemic and other pathogenic outbreaks. In alternative embodiments methods, apparatus and systems as provided herein provide a rapid, high throughput, inexpensive serological test which can:
a) Determine the penetrance, burden, and density of infected people including asymptomatic individuals to enable surveillance, containment and mitigation of COVID-19,
b) Diagnose COVID-19 and differentiate among different respiratory infections
b) Allow identification of individual's immunity to COVID-19 to allow people including healthcare workers to safely perform otherwise high-risk tasks,
c) Help restart the economy in a controlled way minimizing the risk of further waves of infections,
d) Identify donors with potent functional antibodies enabling passive immunization through plasma transfusion, and/or
e) be used as a research tool to provide insights to SARS-CoV-2 infection and informs antigen selection and design diagnostic assays and therapeutic and vaccine development, and to correlate antibody responses with clinical outcomes.

In alternative embodiments, methods, apparatus and systems as provided herein enable analysis of environmental samples, food, meats, beverages, and water. In alternative embodiments, methods, apparatus and systems as provided herein enable analysis and counting of yeast cells for brewing and wine industries. In alternative embodiments, methods, apparatus and systems as provided herein enable analysis and counting of somatic cell count (SCC) in milk.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Exemplary Products of Manufacture and Methods for Using them

For point-of-care COVID-19 serology testing, the said antigen microarray slides are probed with human sera, plasma, or whole blood in appropriate media. Blood samples can be collected using apheresis collection, a syringe through venipuncture or a finger prick (or fingerstick) device. Plasma samples can be prepared from blood using a centrifuge or a microfluidic device (for example centrifugal microfluidic biochip, or CD microfluidics). For serum, blood is allowed to clot prior to separation by centrifugation. As few as one drop of blood (or approximately 20 μl) will be sufficient for COVID-19 testing. These samples are reacted with the said microarray for a period of time ranging from 5 min to overnight at room temperature or 4° C. A washing step can be performed prior to labeling with (secondary) antibodies to human IgA and IgG conjugated to dyes, fluorophores (for example, Cy3, Alexa647) or quantum dots (for example, QD585) for another period of time ranging from 5 min to 6 hours at room temperature. A final washing step can be performed with appropriate buffer to remove any unbound moieties. This entire process can be performed manually by a technician or be automated. The final sides can be analyzed immediately or dried and stored until analysis using our disclosed systems as described above. The microarray consisted of 2×8 pads of 6.5 mm×6.5 mm each. 2×4 pads were imaged simultaneously with 10 μm spatial resolution to resolve the individual dots (100-300 μm) of the microarray. A 2×8 microarray can read by taking two images. The slide holder will maintain to position of the microarrays at the appropriate distance to the camera lens during image acquisition/reading of the slides. A suitable image sensor (for example, 5-megapixel color camera with an OMNIVISION OV5647™ sensor) in combination with excitation and emission filter combinations suitable for the fluorophores of the microarray (see Table 1) will be used. Microarray images will be acquired and processed as shown in FIG. 2, FIG. 3, and Example 2. A barcode located on the slide will be also read with the camera to identify the slide and store this information with the images. The median intensity in each spot indicative of antibody levels to the corresponding antigens will be measured and quantified for each spot as illustrated in FIG. 2E.

### Example 2: Exemplary Microarray Fluorescence Imaging Device and Data Analysis

A 3D CAD/CAM exemplary model was designed as depicted in FIG. 1C. After 3D printing of the model, all relevant optical components were inserted and attached. A photograph of the 3D-printed and assembled device together with a microarray slide inserted into the imaging slot and single board computer (RASPBERRY PI 4™) is shown in FIG. 1D. Light from a 2×4 LED array (3 W) mounted at the bottom was guided through 6-mm holes/channels to the individual pads of the microarray. The microarray consisted of 2×8 pads of 6.5 mm×6.5 mm each. 2×4 pads were imaged simultaneously with 10 μm spatial resolution to resolve the individual dots (100-300 μm) of the microarray. The whole 2×8 array was read by taking two images. The slide holder was designed to position the microarrays at the appropriate distance to the camera lens, here 40 mm. Bump stops on each end of the slot ensured the correct position of the slide to reliably image all pads (pads 1-8 followed by pads 9-16). A 5-megapixel color camera with an OMNIVISION OV5647™ sensor, coupled to a lens for a field of view of 16 mm×32 mm, and a 570-nm long pass filter specific to Alexa 594, Alexa647, QD585, QD800, Cy3 and Cy5 was used for fluorescence imaging of microarrays, other example illumination/filter combinations are shown in Table 1. The camera was controlled by a RASPBERRY PI 4™ single board computer to take images of the slide from the top. By exciting and detecting using different light paths, no dichroic mirror for separation of the fluorescence light was needed. Example microarray images acquired and processed with the exemplary device as provided herein and described in these examples are shown in FIG. 2. By taking a reference image of a checkerboard pattern with 7×14 squares, the spherical aberrations were measured and corrected in the microarray images using the Computer Vision (CV) Package for RASPBERRY PI™. Background was subtracted to eliminate spatial variations of the signal offset by high pass filtering with a 20 pixels cutoff, an example is shown in FIG. 2B-C. Pad identification and alignment was performed by calculating the correlation of the known alignment pattern with the image of each pad, an example is shown in FIG. 2D. The median intensity was measured and quantified for each spot as illustrated in FIG. 2E.

### Example 3: Benchmarking of the Microarray Imaging Performance with a Respiratory Virus Antigen Microarray

We designed, 3D printed, and assembled a version of the imager optimized to read the exemplary coronavirus antigen microarray slides as provided herein. A set of 8 LEDs of 365-nm were used with a battery-powered driver circuit (3 W). Test slides with ALEXAFLUOR 647™, Quantum Dots QD585 and QD655, and Cy3 and Cy5 dyes, were probed. Images (FIG. 2 and FIG. 4) showed that a $20, 5-megapixel camera (OMNIVISION OV5647™ sensor) has enough spatial resolution and sensitivity to reliably read microarrays. In comparison with data from a $10,000+ commercial imager we obtained a correlation of over 85% even with our first, unoptimized design. This shows that it is possible to use our low-cost design instead of expensive, bulky, lab-based microscopes.

A respiratory virus antigen microarray for serological testing was printed (https://doi.org/10.1101/2020.04.15.043364) and probed with serum samples and anti-IgG-QD800. Exemplary microarray images of 8 antigen arrays are shown in FIG. 6, all dots can be resolved with high signal-to-noise ratio.

### Example 4: Characterization of the Lower Limit of Detection of an Exemplary Microarray Imaging Device

A working prototype was constructed and several measurements were performed to demonstrate the capabilities exemplary products of manufacture as provided herein.

Specifically, a calibration slide with spots of Cy5 of decreasing concentrations was used to determine the lower limit of detection of the microarray imaging prototype. Fluorescence images of the calibration pattern can be seen in FIG. 7. The Cy5 concentrations are indicated for each column on the top of the image. The lower limit of detection was defined as spots that can be detected with a signal-to-background ratio of greater than (>) 3, in the resulting image corresponding to less than (<) 18 Cy5 molecules/μm².

### Example 5. Detection of Fluorescent Particles in Solution

To the detection of particles in solution with light sheet illumination, we filled sample cuvettes with yellow-green fluorescent latex particles of various sizes (100 nm, 200 nm, 500 nm) suspended in water, placed them in into the sample holder and subjected them to imaging with the cuvette-based mobile imager with light sheet illumination. For fluorescence excitation, 445-nm laser light was used and the fluorescence light was collected through a KODAK WRATTEN NO 55™ filter via the mobile device camera (here: iPhone 6S). Videos of 1920×1080 pixels were recorded at 29.9 frames per second. Regions of 512×512 pixels were extracted and subjected to iMSD analysis, example images are shown in FIG. 11A-C. Single particles can be clearly identified due to the high signal-to-noise ratio and optical sectioning of the light sheet. Since the particles were suspended in a solution, they moved depending on temperature, particle size and solution viscosity according to the diffusion law. To measure the diffusion coefficients, we applied image mean square displacement analysis (iMSD) to the data recorded. Per sample, a total of 512 frames in a region of 512×512 pixels were analyzed, the resulting data are shown in FIG. 11D-F. As expected, a decrease in particle diameter of a factor of two results in doubling of the diffusion coefficient.

### Example 6. Portable Detection of Microorganisms

As an example biomedical assay for the detection of pathogens we prepared a solution of bacteria (B. subtilis) in minimal medium and stained the membrane fluorescently with FM4-64™ dye. Videos of 1920×1080 pixels were recorded at 29.9 frames per second. Regions of 256×256 pixels were extracted and subjected to iMSD analysis, an example image is shown in FIG. 13A. Using an exemplary device as provided herein, single bacteria can be clearly identified due to the high signal-to-noise ratio and optical sectioning of the light sheet. To measure the bacteria motility, we applied image mean square displacement analysis (iMSD) to the data recorded. Per sample, a total of 512 frames in a region of 256×256 pixels were analyzed, the resulting data are shown in FIG. 13B. The MSD shows a non-linear, quadratic dependence as a function of lag time indicating active motion of the live bacteria. Heat shock treatment was then applied to the sample (1 min at 65° C.), followed by image series acquisition with the same parameters, an example image is shown in FIG. 13C. Again, we applied image mean square displacement analysis (iMSD) to the data recorded to measure the bacteria motility and found only passive diffusion, see FIG. 13D, linear dependence of the iMSD. This ability to distinguish between live and dead bacteria could enable antibiotic susceptibility testing.

### REFERENCES

[1] Breslauer, D. N. et al., Mobile Phone Based Clinical Microscopy for Global Health Applications, PLoS One 4, 6320 (2009).
[2] Wei, Q., et al., Fluorescent imaging of single nanoparticles and viruses on a smart phone. ACS Nano 7, 9147-9155 (2013).
[3] Yelleswarapu, V. R., et al., Ultra-high throughput detection (1 million droplets per second) of fluorescent droplets using a cell phone camera and time domain encoded optofluidics. Lab Chip 17, 1083-1094 (2017).
[4] Sung, Y., et al., Open-source do-it-yourself multi-color fluorescence smartphone microscopy. Biomed Opt Express 8, 5075-5086 (2017).
[5] Zhu, H., et al., Wide-field fluorescent microscopy on a cell-phone. Conf Proc IEEE Eng Med Biol Soc, 6801-6804 (2011).
[6] Khan S., et al. Analysis of Serologic Cross-Reactivity Between Common Human Coronaviruses and SARS-CoV-2 Using Coronavirus Antigen Microarray, Prometheus Study Group. bioRxiv 2020.03.24.006544.
[7] Rafael R. et al, Analysis of SARS-CoV-2 Antibodies in COVID-19 Convalescent Plasma using a Coronavirus Antigen Microarray. bioRxiv 2020.04.15.043364.

A number of embodiments of the invention have been described. Nevertheless, it can be understood that various modifications may be made without departing from the spirit and scope of the invention. Accordingly, other embodiments are within the scope of the following claims.

1. A product of manufacture, a device, an apparatus or a system for testing for the presence of a target analyte in a sample,
wherein optionally the system is a multiplexed system,
and optionally the sample is derived or taken from an individual in need thereof,
and optionally the target analyte comprises a disease biomarker, a protein, a cytokine, a biological particle or a cell,
and optionally the disease biomarker, protein, cytokine or biological particle is expressed by or on or is derived from a cell,
and optionally the target analyte comprises at least one or a plurality of viral or microbial antigens, optionally coronavirus antigens, optionally SARS-CoV-2 antigens or antigen variants,
and optionally the sample comprises a biological or clinical sample, and optionally the biological or clinical sample comprises a liquid sample, and optionally the liquid sample comprises a serum sample,
the product of manufacture, device, apparatus or system comprising:
(a) a functionalized surface, an assay surface or a sample slide,
wherein optionally the functionalized surface or sample slide comprises or is fabricated as a biochip, a microarray or an equivalent, or the functionalized surface is on or fabricated as a bead or a microbead or an equivalent,
and optionally the functionalized surface or sample slide comprises an assay surface that displays or has affixed thereon or carries: at least about 2, at least about 3, at least about 4, at least about 5, at least about 6, at least about 10, at least about 20, at least about 50, at least about 100, at least about 200, at least about 1,000, at least about 10,000, or more, discrete areas or spots, and each discrete area or spot comprises, displays, has affixed thereon or carries: a distinct target analyte-binding composition, or a plurality of compositions each capable of specifically binding to a specific target analyte,
wherein optionally the distinct target analyte-binding composition comprises a protein, and optionally the protein comprises an antibody or antigen binding fragment thereof;
(b) an imaging system capable of imaging a sample in a solution or on a hard surface comprising:
(i) a sample illumination or sample luminescence, fluorescence or phosphorescent excitation device capable of or fabricated to produce an excitation light or excitation beam,
wherein optionally the sample illumination or sample luminescence, fluorescence or phosphorescent excitation device comprises or is fabricated as an epi-illumination-based slide imager, or, a light sheet-based solution imager;
wherein optionally an illumination pattern made by the sample illumination or the sample luminescence, fluorescence or phosphorescent excitation device comprises a round or a rectangular shape to provide uniform epi-illumination of a surface area,
and optionally the surface area is about 5×5 mm to about 50×50 mm, or, in a solution, the illumination pattern is in the form of a light sheet between about 1 μm to about 15 μm thickness to provide optical sectioning, and optionally to provide optical sectioning in an area of about 0.5×0.5 mm to about 5×5 mm,
(ii) a camera or equivalent or a two-dimensional (2D) sensor or equivalent capable of focusing on or capturing images of or detecting light emitted from the two-dimensional (2D) sensor or equivalent and the functionalized surface, an assay surface or a sample slide;
and optionally the 2D sensor or equivalent is a high resolution sensor, and optionally the high resolution sensor has a capability of about 1,000×1,000 pixels to about 10,000×10,000 pixels,
and optionally the 2D sensor or equivalent is a monochrome or color complementary metal-oxide-semiconductor (CMOS) sensor, and optionally the CMOS sensor provides frame rates of about 1 frame per second to about 100 frames per second,
(iii) one or a plurality of lenses for image magnification and focusing of the excitation beam aligned between the camera or equivalent or the two-dimensional (2D) sensor or equivalent and the functionalized surface, an assay surface or a sample slide;
one or a plurality of optical filters to define excitation and detection spectral windows; and,
(iv) a sample holder,
wherein the functionalized surface or sample slide is loaded or positioned onto or into the sample holder or cuvette or equivalent,
and the sample illumination or sample luminescence, fluorescence or phosphorescent excitation are provided by one or multiple light sources in the form of epi-illumination or in the form of a light sheet,
and luminescent, fluorescence or phosphorescent images are detected with the camera or 2D sensor equivalent,
wherein optionally the sample holder comprises or is fabricated as a slide holder or a cuvette or equivalent,
and optionally the excitation light has between about 1 to 100 nm bandwidth,
and optionally the excitation light bandwidth is a narrowband laser light, wherein optionally the narrowband laser light has about a less than (<) 5 nm bandwidth,
and optionally the excitation light comprises a light-emitting diode (LED) light or a light emitting diode,
and optionally the LED light(s) or light emitting diode(s) emit in a specific wavelength range, and optionally the specific wavelength range is in a wavelength range of between about 460 nm to 470 nm, 520 nm to 530 nm or 620 nm to 630 nm,
and optionally emitters of different wavelengths are combined in the same light emitting diode, and optionally the light emitting diode comprises or is a Red, Green, Blue (RGB) emitter,
and optionally the LED light or light emitting diode comprises one or a plurality of spectral filters,
and optionally the one or the plurality of spectral filters have a bandwidth of between about 5 nm to about 100 nm (to result in a narrower spectral band),
and optionally the camera or 2D sensor is operatively connected to a lens or system or plurality of lenses to relay and magnify the image,
and optionally the camera or 2D sensor is operatively coupled to one or multiple or plurality of optical filters to define or to narrow detection of or detected spectral band or bands,
and optionally the camera or 2D sensor or equivalent is or is fabricated as a mobile camera or a 2D sensor equivalent device, or a dedicated camera or 2D sensor equivalent,
and the camera or 2D sensor equivalent is operatively connected to a computer, a smartphone, or equivalent,
and the imaging system, or the camera or 2D equivalent, is operatively linked to a firmware and/or software or computer program to store, process and/or transmit data representing images from the camera or 2D equivalent; and
(c) a firmware and/or software or computer program to acquire, store and process images from the imaging system in a manner suitable for storing, identifying, quantifying and optionally classifying target analytes.

2. The product of manufacture, device, apparatus or system of claim 1, further comprising a step:
(d) a software and/or a computer program to visualize, analyze, share and/or store data representing the images, and optionally the software and/or the program visualizes, analyzes, shares and/or stores the data on the product of manufacture, device, apparatus or system itself or by connecting the product of manufacture, device, apparatus or system to a server or cloud computing-based storage and/or sharing system.

3. The product of manufacture, device, apparatus or system of claim 1, wherein the imaging system, and imaging detection, comprises an about 0.2 to 200 megapixels charge-coupled device (CCD) or a Complementary Metal Oxide Semiconductor (CMOS) color or monochrome camera coupled to a lens or lens system of between about 1 mm to about 1400 mm focal length to yield a field of view of between about 1 mm to about 75 mm with a pixel size at the sample of between about 0.1 μm to 100 μm for 2D imaging of: surfaces with epi-illumination, or solutions with light sheet illumination,
and optionally a fluorescent, luminescent or phosphorescent light is filtered with a long pass, band pass or short pass emission filter to prevent excitation light from reaching the camera sensor.
and optionally the emission light is separated with multiple optical filters into several color channel for multiplex imaging, and optionally one or multiple of the optical filters have a transmission response in the shape of a sine or cosine function or linear ramp to spectrally resolve the detected signal,
and optionally the emission light is separated with a dispersive element into a plurality of spectral channels to spectrally resolve the signal,
and optionally the dispersive element comprises a prism or a grating,
and optionally the emission light is separated with a dispersive element into between about 4 and about 1,000 spectral channels,
and optionally the excitation light is modulated and the emission light is detected in a time-resolved fashion or demodulated to separate components of different luminescence, fluorescent or phosphorescent lifetimes.

4. The product of manufacture, device, apparatus or system of claim 1, wherein the slide holder or cuvette or equivalent comprises a slot or slide insert to accurately position the sample slide, biochip or microarray for imaging,
wherein optionally the slide holder or cuvette or equivalent can accurately position the slide, biochip or microarray at a precision higher than about 1 mm,
and optionally the sample slide, biochip, microarray or cuvette or equivalent can be contained in a cassette or sled to adapt to contain or hold slides, biochips, microarrays, cuvette or equivalent of different sizes to the same imaging device,
and optionally the sample slide, biochip, microarray or cuvette or equivalent have dimensions of between about 1 mm to 5 mm to about 100 mm to 120 mm,
and optionally a cassette or sled is used to position and image between about 1 to about 20 slides at a time on a single device,
and optionally a cassette or sled is used to position and image between about 1 to about 20 slides in between about 1 to 15 min,
and optionally the sample slide, biochip, microarray or cuvette or equivalent is moved through the imaging device either manually, semi-automated and/or fully automated.

4. The product of manufacture, device, apparatus or system of claim 1, wherein the sample slide is illuminated by one or more light sources from the top, bottom and/or side at an angle of between about 0° to 90° to the optical axis or with a light sheet at an angle of between about 60° to 120° to the signal detection axis with the one or multiple light sources,
and optionally the one or multiple light sources comprise or emit visible, near ultraviolet (UV) and/or near infrared (IR) light,
and optionally the visible light comprises light at between about 400 nm to about 700 nm, the near UV comprises light at between about 250 to about 400 nm, and/or near IR comprises light at between about 700 to about 1300 nm,
and optionally the one or multiple light sources are generated using one or more LEDs and/or laser diodes.

6. The product of manufacture, device, apparatus or system of claim 5, wherein illumination comprises or the light source emits epi-illumination, or the illumination comprises or the light source emits light in the form of a sheet or single plane of light at the sample plane,
wherein optionally the sheet or single plane of light at the sample plane is between about 1 μm to 15 μm in thickness,
and optionally the illumination is formed with a cylindrical optical element or by beam scanning,
and optionally the illumination comprises broad band excitation light that is spectrally cleaned with one or more optical filters,
and optionally the one or more optical filters comprises short pass or band pass filters,
and optionally the short pass or band pass filters have between about a 1 nm to about 100 nm bandwidth,
and optionally the source of the illumination or the light source comprises one or multiple LEDs or lasers,
and optionally between about 1 to 100 LEDs or lasers are used,
and optionally between about 1 to 100 LEDs or lasers with different wavelengths are used for multiplex acquisitions in multiple color channels,
and optionally between about 1 to 20 or 2 to 10 different wavelengths are emitted by the LEDs or lasers,
and optionally the light source and/or detector are modulated or pulsed for time-resolved signal detection to increase sensitivity or multiplexing applications,
and optionally time-resolved measurements comprise fluorescence and/or phosphorescence lifetime detection,
and optionally a modulation/pulse frequency for fluorescence lifetime detection is between about 1 to about 100 MHz,
and optionally a modulation/pulse frequency for phosphorescence lifetime detection is between about 1 Hz to about 1 MHz.

7. The product of manufacture, device, apparatus or system of claim 1, wherein the components of the imaging system are designed or fabricated to image surfaces, slides, biochips, microarrays or other target analytes in a sample or solution stained with a detectable dye or particle,
and optionally the detectable dye or particle comprises a fluorescent dye or particle,
and optionally the detectable dye or particle is conjugated to an antibody,
and optionally the antibody comprises a detecting antibody, a secondary antibody or an antibody comprising a streptavidin/biotin linkage,
and optionally the detectable dye or particle comprises: DyLight 405, Cy2, Cy3, Cy5, Alexa488, Alexa546, Alexa594, Alexa647, Atto488, Atto550, Atto647 or an equivalent or a combination thereof.
and optionally the detectable particle comprises: a QD525, QD565, QD585, QD605, QD655, QD705, QD800 nm emission ultrabright fluorescent microsphere or an equivalent or a combination thereof.

8. The product of manufacture, device, apparatus or system of claim 1, wherein the firmware and/or software or computer program used to acquire, store and process images from the imaging system in a manner suitable for storing, identifying, quantifying and optionally classifying target analytes comprises a firmware and/or computer software to interface with the camera or 2D imaging equivalent,
and optionally the firmware and/or software comprises an element or is able to: adjust the camera exposure time and/or sensitivity setting, and/or to correct for image distortions by referencing with an internally stored calibration pattern, to subtract background to eliminate spatial variations of the signal offset, and/or to find the position of each spot of a microarray image by aligning to reference markers printed on the microarray slides, and/or to optimize the dynamic range and ensure a linear response,
and optionally the camera is adjusted to have an exposure time between about 1 microsecond (μs) and 10 seconds (s),
and optionally the camera is adjusted to have a sensitivity setting between about ISO 64 to about ISO 128,000.

9. The product of manufacture, device, apparatus or system of claim 1, wherein a barcode attached or printed on the sample slide or cuvette or equivalent,
and optionally the barcode is read with the camera or with an external reader,
and optionally the barcode read is stored with an image or images of each sample for unambiguous sample identification.

10. The product of manufacture, device, apparatus or system of claim 1, wherein a wired (optionally Ethernet, USB) or wireless connection (optionally Bluetooth, WiFi or equivalents) or equivalent is used to transfer image data to an online server and/or a cloud-based system.

11. The product of manufacture, device, apparatus or system of claim 1, wherein the processing software comprises data comprising the computation of fluorescence, luminescence or phosphorescence intensity values for each spot or particle on a surface or in a 2D plane within a solution by calculating the mean or median value of the intensity distribution within the spot area,
and optionally the calculating comprises referencing the data to a known antigen layout of the microarray, biochip or sample slide or composition of the capture beads or microbeads,
and optionally attached dyes with known concentrations as internal reference, probability values (p values) for the presence of target analytes specific to a disease can be calculated,
and optionally the referencing step is carried out either on the device itself or on a server/cloud-based system after data upload.

12. The product of manufacture, device, apparatus or system of claim 1, wherein the distinct target analyte comprises a distinct protein,
and optionally distinct protein is an antibody or an antigen,
and optionally the antigen is derived from a microorganism, and optionally the microorganism is a bacterium or a virus, and optionally the viral antigen comprises a SARS-CoV-2 antigen or antigenic variant,
and optionally the antibody specifically binds to a microorganism, and optionally the microorganism is a bacterium or a virus, and optionally the viral antigen comprises a SARS-CoV-2 antigen or antigenic variant,
and optionally the functionalized surface comprises, displays or carries at least 1 coronavirus antigen, and optionally the coronavirus comprises an S protein antigen,
and optionally the coronavirus is or comprises SARS-CoV or MERS-CoV,
and optionally the functionalized surface comprises, displays or carries a non-coronavirus antigen, optionally a viral or a bacterial antigen,
and optionally the viral antigen is or comprises a HKU1, OC43, NL63, 229E or influenza antigen.

13. The product of manufacture, device, apparatus or system of claim 1, wherein, the target analytes are captured or affixed on a surface or the sample slide,
wherein optionally the microarray, biochip, sample slide or bead or microbead is probed with a detectable dye or a nanoparticle, optionally using a sandwich assay,
and optionally the bead or microbead with a captured target analyte is subsequently immobilized on a surface, or a slide, or a microwells, or a microchamber, prior to imaging,
and optionally the bead or microbead with the captured target analytes remains suspended in solution for imaging.

14. The product of manufacture, device, apparatus or system of claim 1, wherein a target analyte is directly labeled with a detectable dye or a nanoparticle prior to immobilizing or affixing onto a surface for analysis, or the target analyte is analyzed directly in solution,
wherein optionally the target analyte is or is derived from: a cell, a cancer cell, a CTC, an immune cell, a bacterium, a yeast cell, a pathogen, a virus or a parasite.

15. The product of manufacture, device, apparatus or system of claim 1, fabricated as a portable device.

16. The product of manufacture, device, apparatus or system of claim 1, wherein the device further comprises or is fabricated to have:
a hollow tubing for flowing or moving a liquid sample, wherein the hollow tubing is operatively linked to a transparent sample flow cell, a pump and optionally an automated axial sample holder, wherein a liquid sample flows through the hollow tubing, the transparent sample flow cell and the pump;
a pump operatively linked to the hollow tubing, wherein flow of a liquid in and through the hollow tubing and the transparent sample flow cell is controlled and driven by the pump,
an automated axial sample holder and a mechano-electric component, wherein movement of the automated axial sample holder is controlled by the mechano-electric component, and the automated axial sample holder positions the transparent sample flow cell before a source of light or illumination or an excitation light or excitation beam,
wherein optionally the automated axial sample holder comprises or is fabricated as a piezo actuator or voice coil actuator;
and optionally the transparent sample flow cell is or is fabricated as a cuvette or equivalent,
and optionally the source of light or illumination is or is fabricated as a sample illumination or sample luminescence, fluorescence or phosphorescent excitation device capable of or fabricated to produce an excitation light or excitation beam,
and optionally in combination with flowing the liquid solution through the transparent sample flow cell (optionally a cuvette or equivalent) a volume of liquid (optionally between about 0.1 ml to about 10 ml liquid) is imaged (optionally in a few minutes) with the ability to detect the presence of a particle (optionally able to detect only a single particle).

17-18. (canceled)

19. A method for detecting one or a plurality of biomarkers, optionally one or a plurality of proteins, optionally one or a plurality of viral or a microbial antigens, optionally one or a plurality of antibodies, optionally one or a plurality of cytokines, optionally one or a plurality of biological particles, optionally one or a plurality of cells, or diagnosing, or optionally measuring immune response to, a viral or a microbial infection, cancer, autoimmune disorder, or inflammation, comprising:
(a) providing of having provided a product of manufacture, device, apparatus or system of claim 1;
(b) contacting the product of manufacture, device, apparatus or system with a biological sample or a sample derived from a biological source comprising a target analyte,
wherein optionally the biological sample or the sample derived from the biological source comprising a target analyte comprises or is derived from a solid tissue sample, a biopsy or a biological liquid, wherein optionally the biological liquid comprises or is derived from a serum, blood, urine, cerebral spinal fluid, mucous or sputum sample;
(c) determining if the biological sample or the sample derived from the biological source comprises a composition (optionally a protein) that specifically binds to a target analyte affixed on the product of manufacture, device, apparatus or system.

20. The method of claim 19, wherein the target analyte comprises or is a substantially stained or detectably labeled target analyte,
wherein optionally the stained or detectably labeled target analyte comprises a fluorescently or luminescently stained target analyte, and optionally the stained or detectably labeled target analyte is in solution or in a liquid form,
and optionally target analytes in a sample or solution are stained with a detectable dye or particle before the target analyte is contacted with or to the product of manufacture, device, apparatus or system,
and optionally the detectable dye or particle comprises a fluorescent dye or particle,
and optionally the detectable dye or particle is conjugated to an antibody,
and optionally the antibody comprises a detecting antibody, a secondary antibody or an antibody comprising a streptavidin/biotin linkage,
and optionally the detectable dye or particle comprises: DyLight 405, Cy2, Cy3, Cy5, Alexa488, Alexa546, Alexa594, Alexa647, Atto488, Atto550, Atto647 or an equivalent or a combination thereof,
and optionally the detectable particle comprises: a QD525, QD565, QD585, QD605, QD655, QD705, QD800 nm emission ultrabright fluorescent microsphere or an equivalent or a combination thereof,
and optionally the determining if the biological sample or the sample derived from the biological source comprises using a composition, and optionally the composition comprises a protein, that specifically binds to a specific target analyte affixed on the product of manufacture, device, apparatus or system, and the composition comprises a substantially stained or a detectable agent or moiety that can specifically bind to a target analyte that is specifically bound to an assay surface of the product of manufacture, device, apparatus or system, and optionally the composition is detectably stained, and optionally the composition is fluorescently stained, and optionally the composition is in solution or in a liquid form.

21. (canceled)

22. A method for treating or ameliorating a disease, a condition, or a viral or a microbial infection, comprising:
(a) testing or screening an individual in need thereof with a product of manufacture, device, apparatus or system of claim 1, to determine if the individual in need thereof is infected with a pathogen, a virus or a microbe, or has a disease or condition, and
(b) and if the individual in need thereof is found to be infected with the pathogen, the virus or microbe, or is determined to have or is diagnosed with the disease or condition, administering a drug or a treatment or agent for ameliorating or decreasing the symptoms of the disease, condition, pathogen virus or microbe, or administering a drug or a treatment to treat or ameliorate or decrease the symptoms of a condition, disease, infection or symptom caused by the pathogen, virus or microbe,
wherein optionally the disease or condition is cancer or a condition comprising inflammation.

23. A kit comprising:
(a) a product of manufacture, device, apparatus or system of claim 1;
(b) at least one set of buffer or media suitable for binding and washing; and
(c) at least one set of detectable dyes or particles;
and optionally further comprising detecting or secondary antibodies conjugated to detectable dyes,
and optionally further comprising a sample collection device or a target analyte processing device,
and optionally the target analyte processing device comprises a component or reagent for enrichment, extraction, purification, labeling, conjugation of the target analyte.
